SEQUENCE LISTING (1 sequence)

SEQ ID NO: 1 - protein, 412 aa, Homo sapiens
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

(19) United States
(12) Patent Application Publication
(10) Pub. No.: US 2025/0000859 A1
(43) Pub. Date: Jan. 2, 2025

(54) USE OF GCN2 INHIBITORS IN TREATING CANCER

(71) Applicant: Alesta Therapeutics BV, Leiden (NL)

(72) Inventors: Sandra SCHOORS, Mechelen (BE); Matthias VAN WOENSEL, Westerlo (BE)

(21) Appl. No.: 18/686,359

(22) PCT Filed: Aug. 25, 2022
(86) PCT No.: PCT/EP2022/073727

§ 371 Date: Feb. 23, 2024

Related U.S. Application Data
(60) Provisional application No. 63/237,091, filed on Aug. 25, 2021.

(51) Int. Cl. A61K 31/505; A61K 31/416; A61K 31/4184; A61K 31/47; A61K 31/4704; A61K 31/4706; A61K 31/4709; A61K 31/4725; A61K 31/498; A61K 31/4985; A61K 31/506; A61K 31/517; A61K 31/519; A61K 31/52; A61K 31/5383; A61P 35/00; C12Q 1/6886; G01N 33/574

(52) U.S. Cl. CPC A61K 31/505; A61K 31/416; A61K 31/4184; A61K 31/47; A61K 31/4704; A61K 31/4706; A61K 31/4709; A61K 31/4725; A61K 31/498; A61K 31/4985; A61K 31/506; A61K 31/517; A61K 31/519; A61K 31/52; A61K 31/5383; A61P 35/00; C12Q 1/6886; G01N 33/57484; C12Q 2600/158; G01N 2333/9015

(57) ABSTRACT

Provided herein are methods of treating cancers that express a low level of a urea cycle enzyme with a GCN2 inhibitor. Also provided herein are diagnostic methods comprising detecting a urea cycle enzyme. Kits comprising a GCN2 inhibitor and instructions for detecting a urea cycle enzyme are also provided herein.

### CROSS REFERENCE TO RELATED APPLICATIONS

This application claims the benefit of, and priority to, U.S. Provisional Application No. 63/237,091 filed Aug. 25, 2021, the contents of which are incorporated herein by reference in its entirety.

### REFERENCE TO AN ELECTRONIC SEQUENCE LISTING

The contents of the electronic sequence listing (232912000740SEQLIST.xml; Size: 2,275 bytes; and Date of Creation: Aug. 22, 2022) is herein incorporated by reference in its entirety.

### FIELD OF INVENTION

The present invention relates to the use of GCN2 inhibitors to treat cancers with low levels or activity of a urea cycle enzyme(s).

### BACKGROUND

Arginine is an amino acid involved in numerous biological processes including: cell proliferation, cell signaling, immunity, neurotransmission, and synthesis of growth factors and other amino acids. Three major sources of arginine include: dietary intake from arginine-enriched nutrition supplements, endogenous synthesis from citrulline, and protein catabolism

Arginine is also considered a “semi-essential” or “conditionally essential” amino acid. Normal cells have an intrinsic ability to synthesize arginine from citrulline and aspartate via argininosuccinate synthase 1 (ASS1, ASS, CTLN1) and other urea cycle enzymes. Whereas normal cells do not depend on external arginine, many cancer cells downregulate the urea cycle via, e.g. ASS1 downregulation, and are dependent on the external arginine. ASS1-low tumors are commonly associated with chemoresistance and poor clinical outcomes.

In mammalian cells, the Arginine deprivation leads to stress and activation of a widely conserved signalling pathway termed the Integrated Stress Response (ISR) pathway. The activation of ISR can trigger cell-cycle arrest, differentiation, amino acid biosynthetic and transport pathways, compensatory adaptation (ADD), or apoptosis, depending on the cell type and the initiating stress.

Nonderepressible 2 (GCN2) is one of four stress kinases of the ISR pathway that senses amino acid availability and controls gene expression in response to amino acid deprivation, UV-irradiation, viral infection, proteasome inhibition, hypoxia, glucose deprivation, and oxidative stress. In mammals, GCN2 is also called EIF2AK4 (eukaryotic translation initiation factor 2 alpha kinase 4).

GCN2 inhibitors have been identified as promising cancer therapeutics. However, some cancers may be more responsive to GCN2 inhibitors depending on additional mutations or metabolic deficiencies. Thus, there is a need to identify cancers that are particularly responsive to GCN2 inhibitors for more targeted treatment.

### SUMMARY OF THE INVENTION

Provided herein is a method of treating a cancer in an individual comprising administering a GCN2 inhibitor to the individual, wherein the cancer has a low level of expression or activity of a urea cycle enzyme.

In some embodiments, provided herein is a method of treating cancer in an individual comprising (a) detecting the level of expression or activity of a urea cycle enzyme, and (b) administering a GCN2 inhibitor to the individual if the cancer expresses a low level of expression or activity of a urea cycle enzyme.

Also provided herein is a method of predicting responsiveness to a GCN2 inhibitor in an individual having cancer comprising detecting the level of expression or activity of a urea cycle enzyme in the cancer, wherein if the cancer has a low level of expression or activity of a urea cycle enzyme, the cancer is responsive to the GCN2 inhibitor.

In some embodiments, provided herein is method of inhibiting tumor growth in an individual comprising administering a GCN2 inhibitor to the individual, wherein the tumor has a low level of expression or activity of a urea cycle enzyme.

In some embodiments, provided herein is a method of inhibiting cell proliferation in an individual comprising administering a GCN2 inhibitor to the individual, wherein the cell has a low level of expression or activity of a urea cycle enzyme.

In some embodiments, an effective amount of a GCN2 inhibitor is administered.

In some embodiments, the urea cycle enzyme is ASS1. In some embodiments, the cancer, tumor, or cell expresses a low level of ASS1 protein or mRNA.

In some embodiments, the method further comprises comparing the level of expression or activity of ASS1 to a control. In some embodiments, the cancer, tumor, or cell has at least 1.5 fold lower expression of ASS1 than the control. In some embodiments, the control is a sample is obtained from non-cancerous tissue of the same origin as the cancer, tumor, or cell. In some embodiments, the control is the average expression level of ASS1 level derived from a population of subjects.

In some embodiments, the GCN2 inhibitor is a compound of formula (I), (I-1), (1-2), (II), (III) or (IV), a pharmaceutically acceptable salt, stereoisomer or tautomer thereof. In some embodiments, the GCN2 inhibitor is selected from the group consisting of Compounds 1-8.

In some embodiments, the GCN2 inhibitor decreases the activity of PERK. In some embodiments, the activity of PERK is decreased at least 1.5 fold.

In some embodiments, the GCN2 inhibitor decreases the level of CHOP. In some embodiments, the level of CHOP is decreased at least 1.5 fold.

In some embodiments, the GCN2 inhibitor does not decrease the activity of PERK. In some embodiments, the GCN2 inhibitor does not decrease the level of CHOP.

In some embodiments, the cancer is a solid or hematological tumor. In some embodiments, the cancer is selected from the group consisting of breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, adenocarcinoma, hepatocellular carcinoma, thyroid cancer, multiple myeloma, cancer of secretory cells, myelodysplastic syndrome, myeloproliferative neoplasm, malignant glioma, non-Hodgkin's lymphoma, Hodgkin's lymphoma, Burkitt's lymphoma, chronic lymphocytic leukemia, chronic myeloid leukemia, hairy cell leukemia, monoclonal gammopathy of undetermined significance (MGUS), plasmacytoma, lymphoplasmacytic lymphoma, acute lymphoblastic leukemia, acute myeloid leukemia, chronic myelomonocytic leukemia, juvenile myelomonocytic leukemia, large granular lymphocytic leukemia, B-cell prolymphocytic leukemia, T-cell prolymphocytic leukemia, small cell lung cancer, malignant pleural mesothelioma, Head and neck squamous cell carcinoma, glioblastoma multiforme, sarcoma, or pediatric neuroblastoma.

In some embodiments, the level of expression of the urea cycle enzyme is determined by measuring mRNA level or protein level. In some embodiments, the level of ASS1 protein or mRNA is measured by western blot or immunohistochemistry, qPCR, FISH, nanostring, or RNAseq.

In some embodiments, the level of ASS1 protein expressed by the cancer is lower than the level of ASS1 protein expressed in non-cancer cells or less responsive cancers.

In some embodiments, the tumor environment comprises a low level of arginine.

In some embodiments, the individual is a human.

Also provided herein is a kit comprising a GCN2 inhibitor and instructions for detecting the level of a urea cycle enzyme. In some embodiments, the kit further comprises an agent for detecting the level of the urea cycle enzyme. In some embodiments, the agent is an antibody. In some embodiments, the antibody is detectably labeled.

All references provided herein are incorporated by references in their entireties.

### BRIEF DESCRIPTION OF THE DRAWINGS

FIG. 1 shows the EC50 of GCN2 inhibitors on murine 3T3 cells stably transduced with a CHOP reporter. Cells were stimulated with 100 nM halofuginone or 200 nM tunicamycin, in the presence of increasing concentrations of test compounds. The Y-axis shows the EC50 for halofuginone and the X-axis shows that EC50 for tunicamycin. Provided are representative results (mean) of at least 3 independent experiments.

FIG. 2A shows the EC50 of GCN2 inhibitors on CHOP reporter cells stimulated with 1 uM halofuginone in presence of increasing concentrations of test compounds. Provided are the results of 4 independent experiments.

FIG. 2B shows the EC50 of GCN2 inhibitors on HT29 and HCT116 cells stably transduced with an ATF4 reporter. ATF4 reporter cells were stimulated with halofuginone in the presence of increasing concentrations of test compounds. Provided are the results of 2 independent experiments.

FIG. 2C shows the combined EC50 of the GCN2 inhibitors for CHOP and ATF4.

FIG. 3A shows ASS1 expression in colorectal cancer cell lines HT29 and HCT116. Cells were cultured in a medium before samples were harvested and analyzed via Western blot. Alpha-actinin was used as a loading control.

FIG. 3B shows the relative viable spheroid size and morphology of HT29 cells following treatment with Compound 1. Plates were scanned every 4 hours for up to 120 hours.

FIG. 3C shows the relative viable spheroid size of HCT116 cells following treatment with Compound 1. Plates were scanned every 4 hours for up to 120 hours.

FIG. 3D shows the relative size of the HT29-based spheroids treated with a dose-response of indicated compounds compared to control.

FIG. 3E shows the relative size of the HCT116-based spheroids treated with a dose-response of indicated compounds compared to control.

FIG. 4 shows the EC50 values of indicated test compounds on the growth of HT29-base spheroids. HT29 spheroids were formed for 3 days and treated with a dose-response of indicated compounds.

FIG. 5A shows ASS1 expression in A498 and 769-P renal cell carcinoma cell lines. Cells were cultured in a medium before samples were harvested and analyzed via Western blot. Alpha-actinin was used as a loading control.

FIG. 5B shows the relative viability of renal cell carcinoma lines 769-P and A498 to after treatment with GCN2 inhibitors.

FIG. 6 shows the effect of compound 2 on HT29 and HCT116 cancer cell growth. Compound 2 has an IC50 of 51.42 nM for HT29 cells and an IC50 of 1333 nM for HCT116 cells.

FIG. 7A is a western blot comparing expression of ASS1 in A498 cells and 769P cells. α-tubulin is included as a control. FIG. 7B shows the percent viability of 769P and A498 cells treated with varying amounts of compound 2 compared to untreated cells.

FIG. 8A shows the relative EdU labeling of HT29 cells incubated with 100 μm, 25 μm, 12.5 μm, or 6.25 μm arginine treated with compound 1, compound 2, compound 3, or vehicle FIG. 8B shows the relative EdU labeling of HCT116 cells incubated with 100 μm, 25 μm, 12.5 μm, or 6.25 μm arginine treated with compound 1, compound 2, compound x, or vehicle control.

FIG. 9A is a boxplot showing the inhibition of tumor volume of Renca, CT26, MC38, or B16F10 cells implanted in the flank of BALB/c or C57BL/6 mice treated with 30 mg/kg compound 1 administered orally. Percent inhibition of tumor volume is relative to untreated control. FIG. 9B is a boxplot showing the inhibition of tumor volume of Renca, CT26, MC38, or B16F10 cells implanted in the flank of BALB/c or C57BL/6 mice treated with 20 mg/kg compound 2 administered orally.

FIG. 10A shows ASS1 mRNA expression in primary solid tumors and normal tissue. FIG. 10B shows the Cancer Genome Atlas (TCGA) expression analysis of ASS1 normal and ASS1 low cells. ASS1 low cells comprises cells with the lowest quartile of AS1 expression. Positive numbers indicate a positive correlation between two factors. FIG. 10C is a box plot showing the risk of autophagy of ASS1 low (bottom quartile of ASS1 expression), ASS1 normal (middle two quartiles of ASS1 expression), and ASS1 high (top quartile of ASS1 expression).

### DETAILED DESCRIPTION

In some embodiments, the present invention relates to the surprising finding that cancers with low levels of a urea cycle enzyme are responsive to GCN2 inhibitors. In some embodiments, cancers with low ASS1 levels are more responsive to GCN2 inhibitors than those with normal or high levels of ASS1. Without being bound by theory, in ASS1-low tumors, GCN2 may provide a survival advantage through the activation of protective mechanisms. Therefore, modulation of general control nonderepressible 2 (GCN2) may provide a therapeutic strategy for ASS1-low cancers. Thus, ASS1 or other urea cycle enzymes can serve as useful biomarkers to identify patients suitable for therapy with GCN2 inhibitors.

In addition, the compounds provided herein are presently shown to have unique profiles with respect to GCN2 inhibition and PERK inhibition. For example, a subset of compounds described here selectively inhibit GCN2 but do not inhibit PERK, while others inhibit both GCN2 and PERK. Thus the methods and compounds provided herein can be used for therapy tailored to a patient's particular cancer.

### Definitions

The term “treatment”, in relation to the uses of any of the compounds described herein, including those of Formula (I) is used to describe any form of intervention where a compound is administered to a subject having the disease or disorder in question, such as cancer. Treatment encompasses any one or more of decreasing one or more symptoms resulting from the disease or disorder, diminishing the extent of the disease or disorder, stabilizing the disease or disorder (e.g., preventing or delaying the worsening of the disease or disorder), delaying the recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, increasing the quality of life, and/or prolonging survival of a patient. In some embodiments, treatment does not include prevention.

“Individual” refers to mammals and includes humans and non-human mammals. Examples of individuals include, but are not limited to mice, rats, hamsters, guinea pigs, pigs, rabbits, cats, dogs, goats, sheep, cows, and humans. In some embodiments, individual refers to a human.

The term “sample” means a quantity of a substance. The term “biological sample” means a quantity of a substance obtained from a living thing or formerly living thing. Such substances include, but are not limited to tissues (such as biopsy samples), blood, plasma, serum, urine, amniotic fluid, synovial fluid, endothelial cells, leukocytes, monocytes, other cells, organs, bone marrow, and lymph nodes.

The term “effective therapeutic amount” (for example in relation to methods of treatment of a disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain. With respect to the treatment, if the condition is cancer, then the effective therapeutic amount is an amount sufficient to reduce one or more symptoms associated with cancer, for example reduction of tumour size or rate of metastasis.

As used herein, “about” a parameter or value includes and describes that parameter or value per se. For example, “about X” includes and describes X per se.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Terms such as “heterobicyclic”, “heteroaryl”, “bicyclic”, “heterocyclic”, “carbocyclic”, “alkyl”, “alkoxy” and “halo” are all used in their conventional sense (e.g. as defined in the IUPAC Gold Book), unless indicated otherwise. “optionally substituted” as applied to any group means that the said group may if desired be substituted with one or more substituents, which may be the same or different.

To the extent that any of the compounds described have chiral centres, the present invention extends to all optical isomers of such compounds, whether in the form of racemates or resolved enantiomers. Also provided herein are, where applicable, any and all stereoisomers of the compounds depicted herein, including geometric isomers (e.g., cis/trans isomers or E/Z isomers), enantiomers, diastereomers, or mixtures thereof in any ratio, including racemic mixtures.

The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (+)-DL-lactic), lactobionic, maleic, malic (e.g. (−)-L-malic), malonic, (+)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The term “pharmaceutical composition” in the context of this invention means a composition comprising an active agent and comprising additionally one or more pharmaceutically acceptable carriers that is suitable for administration to an individual. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 μg to about 30 mg per kg body weight of a human and non-human animal, preferably from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

### I. Methods of Treating Low ASS1 Cancers

There are five key enzymes in the urea cycle: carbamoyl-phosphate synthetase 1 (CPS1), ornithine transcarbamylase (OTC), argininosuccinate synthetase (ASS1), argininosuccinate lyase (ASL), and arginase 1 (ARG1). In the first step of the urea cycle, CO and ammonia were converted into carbamoyl phosphate via the enzyme carbamoyl-phosphate synthetase 1. In the second step, ornithine transcarbamoylase catalyzes the donation of the carbamoyl phosphate group to ornithine, producing citrulline. Citrulline is then transported from hepatocyte mitochondria into the cytoplasm by ornithine translocase. In the third step, argininosuccinate synthetase catalyzes condensation reaction between the amino group of aspartate and the carbonyl group of citrulline to form argininosuccinate. In the fourth step, argininosuccinate undergoes cleavage by argininosuccinate lyase to form arginine and fumarate. In the last step, arginine undergoes hydrolysis via arginase to form urea and ornithine. The ornithine is then transported back to the mitochondria to begin the urea cycle again.

Argininosuccinate synthetase 1 (ASS1) (Uniprot: P00966-1, NCBI: XP_011517007) is encoded by ASS1 gene located on chromosome 9 (9q34.11). Argininosuccinate synthetase-1 is a cytosolic urea cycle enzyme mainly expressed in periportal hepatocytes, but also in most other body tissues. The enzyme is a homotetrameric protein composed of 45-kD monomers and is involved in the synthesis of arginine and catalyzes that condensation of citrulline and aspartate to argininosuccinate using ATP. Intratumoral deficiency of ASS1 has been detected in significant numbers of patients with cancers including mesothelioma and NSCLC (Dillon B J et al., Cancer 100:826-833, 2004 and Szlosarek P W et al., Clin Cancer Res 12:7126-7131, 2006). Epigenetic modification via aberrant methylation of the ASS1 promoter is proposed as underlying this deficiency, especially in mesothelioma (Szlosarek P W et al., Clin Cancer Res 12:7126-7131, 2006 and Szlosarek P W et al., JAMA Oncol 3:58-66, 2017). Importantly, low ASS1 expression is associated with a more aggressive clinical phenotype and a worse clinical outcome in several different cancer types (Nicholson L J et al., Int J Cancer 125:1454-1463, 2009; Huang H Y et al., Clin Cancer Res 19:2861-2872, 2013; Allen M D et al. imaging. Cancer Res 74:896-907, 2014; and Lan J et al., Tumour Biol 35:161-169, 2014) Moreover, it is known that patients resistant to the antifolate pemetrexed have high levels of thymidylate synthase and low levels of ASS1 (Allen M D et al. imaging. Cancer Res 74:896-907, 2014).

SEQ ID NO: 1, Human Argininosuccinate synthase
(ASS1)
MSSKGSVVLAYSGGLDTSCILVWLKEQGYDVIAYLANIGQKEDFEEARK
KALKLGAKKVFIEDVSREFVEEFIWPAIQSSALYEDRYLLGTSLARPCI
ARKQVEIAQREGAKYVSHGATGKGNDQVRFELSCYSLAPQIKVIAPWRM
PEFYNRFKGRNDLMEYAKQHGIPIPVTPKNPWSMDENLMHISYEAGILE
NPKNQAPPGLYTKTQDPAKAPNTPDILEIEFKKGVPVKVTNVKDGTTHQ
TSLELFMYLNEVAGKHGVGRIDIVENRFIGMKSRGIYETPAGTILYHAH
LDIEAFTMDREVRKIKQGLGLKFAELVYTGFWHSPECEFVRHCIAKSQE
RVEGKVQVSVLKGQVYILGRESPLSLYNEELVSMNVQGDYEPTDATGFI
NINSLRLKEYHRLQSKVTAK

In some embodiments, ASS1 comprises the amino acid sequence set forth in SEQ ID NO: 1. In some embodiments, the ASS1 comprises one or more insertions, deletions, or substitutions compared to SEQ ID NO:1. In some embodiments, ASS1 comprises a portion of the amino acid sequence set forth in SEQ ID NO:1. In some embodiments, ASS1 is an alternatively spliced variant of SEQ ID NO:1.

In the liver, the urea cycle (UC) converts excess systemic nitrogen, derived from the breakdown of nitrogen-containing metabolites, such as ammonia and glutamine, into urea, a disposable nitrogenous compound. Outside the liver, different UC enzymes are expressed in accordance with cellular needs for UC intermediates. Due to metabolic shifts, many cancer cells come to depend on the presence of exogenous amino acids. For instance, ornithine, which is produced in the last step of the urea cycle, is a precursor for the biosynthesis of polyamines and proline, which are required for a wide variety of cellular functions. Downregulation of urea cycle components, which shunts metabolites away from arginine synthesis and toward pyrimidine biosynthesis to support cell proliferation, is frequently found as part of cancer metabolic reprograming. Studies have shown that the loss of argininosuccinate synthase (ASS1) promotes cancer proliferation by diversion of its aspartate substrate toward carbamoyl-phosphate synthase 2 (CPS2), aspartate transcarbamylase (ATC), and dihydroorotase, the CAD enzyme that catalyzes the first three reactions in the pyrimidine synthesis pathway (Nagamani and Erez, 2016; Rabinovich et al., 2015). The resultant increase in cellular pyrimidines is associated with elevated R/Y transversions at the DNA levels, eliciting a transversion bias (PTMB) on the sense strand that is associated with worse prognosis. PTMB propagates from the DNA to the RNA and protein levels, leading to the generation of peptides with increased predicted immunogenicity (Lee et al., 2018, Cell 174, 1559-1570). Similarly, it was shown that CPS1 maintains the pyrimidine pool in non-small cell lung cancer through CAD activation (Kim et al., 2017).

The basic mechanism of nutritional stress management mediated by GCN2 pathway functions primarily to couple cell growth to amino acid availability (Zhang et al., 2002). In the tumor microenvironment, the abnormal development of vasculature results in insufficient blood supply and deprivation of glucose and amino acids. Both amino acid and glucose deprivation, stresses found in solid tumors, activated GCN2 to upregulate ATF4 target genes involved in amino acid synthesis and transport. GCN2 activation/overexpression and increased phospho-eIF2α were observed in human and mouse tumors compared with normal tissues and abrogation of ATF4 or GCN2 expression significantly inhibited tumor growth in vivo (Ye et al., 2010).

ATF4 is necessary for tumor cells to maintain homeostasis of amino acid metabolism and that activation of GCN2-ATF4-asparagine synthetase (ASNS) pathway promotes tumor cell survival under nutrient (amino acid or glucose) deprivation. GCN2-eIF2α pathway is activated in various human and mouse tumor tissues. Deficiency of ATF4 or GCN2 severely inhibits tumor growth in vivo. Together, these results suggest that GCN2-ATF4-ASNS pathway is a promising target for tumor therapy.

Without being bound to any particular theory, the GCN2-eIF2α-ATF4 pathway is important for maintaining metabolic homeostasis in tumor cells, making it a novel and attractive target for anti-tumor approaches, particularly in the context of tumors that express a low level of a urea cycle enzyme (such as ASS1).

In some embodiments, provided herein is a method of treating cancer comprising administering an effective amount of a GCN2 inhibitor to an individual, wherein the cancer expresses a low level of a urea cycle enzyme. In some embodiments, the cancer expresses a low level of ASS1. In some embodiments, the method comprises treating a solid tumor. In some embodiments, the method comprises treating a hematological cancer. In some embodiments, the method comprises treating a leukemia or a lymphoma.

In some embodiments, the method comprises treating a liver cancer. In some embodiments, the method comprises treating any of breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer (such as HCC), gastrointestinal carcinoma, adenocarcinoma, hepatocellular carcinoma, thyroid cancer, multiple myeloma, cancer of secretory cells, myelodysplastic syndrome, myeloproliferative neoplasm, malignant glioma, non-Hodgkin's lymphoma, Hodgkin's lymphoma, Burkitt's lymphoma, chronic lymphocytic leukemia, chronic myeloid leukemia, hairy cell leukemia, monoclonal gammopathy of undetermined significance (MGUS), plasmacytoma, lymphoplasmacytic lymphoma, acute lymphoblastic leukemia, acute myeloid leukemia, chronic myelomonocytic leukemia, juvenile myelomonocytic leukemia, large granular lymphocytic leukemia, B-cell prolymphocytic leukemia, T-cell prolymphocytic leukemia, small cell lung cancer, malignant pleural mesothelioma, Head and neck squamous cell carcinoma, glioblastoma multiforme, sarcoma, or pediatric neuroblastoma.

In some embodiments, provided herein are methods of treating a liver cancer in an individual comprising administering a compound provided herein to the individual. In some embodiments, provided herein is a method of treating a liver cancer in an individual comprising administering compound 1 or compound 2 to the individual. In some embodiments, the individual has hepatocellular carcinoma (HCC). In some embodiments, the individual has HCC that expresses a low level of ASS1. In some embodiments, the ASS1 level in the HCC is less than 25% of normal tissue.

In some embodiments, provided herein are methods of treating a renal cancer in an individual comprising administering a compound provided herein to the individual. In some embodiments, provided herein is a method of treating a renal cancer in an individual comprising administering compound 1 or compound 2 to the individual. In some embodiments, the individual has renal cell carcinoma (RCC). In some embodiments, the individual has RCC that expresses a low level of ASS1. In some embodiments, the ASS1 level in the RCC is less than 25% of normal tissue.

In some embodiments, provided herein are methods of treating colorectal cancer (CRC) in an individual comprising administering a compound provided herein to the individual. In some embodiments, provided herein is a method of treating a CRC in an individual comprising administering compound 1 or compound 2 to the individual. In some embodiments, the individual has RCC that expresses a low level of CRC. In some embodiments, the ASS1 level in the CRC is less than 25% of normal tissue.

In some embodiments, the cancer is an aggressive cancer. In some embodiments, the cancer expresses one or more markers associated with EMT transition. In some embodiments, the cancer has silenced the ASS1 locus. In some embodiments, the cancer expresses one or more markers associated with autophagy.

Various studies in the literature have shown that ASS1 is deficient in the following tumors: acute myelogenous leukemia (AML), bladder, breast, colorectal, gastric, glioblastoma, liver cancer, lymphoma, melanoma, mesothelioma, non-small cell lung, ovarian, pancreatic, prostate, renal, sarcoma, and small cell lung.

In some embodiments, the cancer is a cancer that expresses a low level of a urea cycle enzyme (such as ASS1), is bladder urothelial carcinoma, breast invasive carcinoma, cholangiocarcinoma, colon adenocarcinoma, glioblastoma multiforme, head and neck squamous cell carcinoma, kidney chromophobe, kidney renal clear cell carcinoma, kidney renal papillary cell carcinoma, liver hepatocellular carcinoma, lung squamous cell carcinoma, prostate adenocarcinoma, rectum adenocarcinoma, or uterine corpus endometrial carcinoma.

In some embodiments, the method comprises measuring the level of a urea cycle enzyme in a sample. In some embodiments, the sample is a cancer cell or cancerous tissue. In some embodiments, the sample is a control sample. In some embodiments the sample is a biopsy. In some embodiments, the sample is a tissue section.

In some embodiments, the method comprises selecting a patient for treatment, wherein the patient has a cancer that expresses a low level of a urea cycle enzyme (such as ASS1) and administering a GCN2 inhibitor to the individual. In some embodiments, the patient is selected using a cutoff value for expression level of the urea cycle enzyme. In some embodiments, the patient is selected using a cutoff value for activity of the urea cycle enzyme. In some embodiments, the patient is selected by comparing the level of expression or activity of a urea cycle enzyme in the cancer and in a control. In some embodiments, the control is non-cancerous tissue. In some embodiments, the control is from a different individual that than the patient. In some embodiments, the control is the average expression or activity level from a collection of individuals.

In some embodiments, the method further comprises comparing the level of a urea cycle enzyme in a cancer to a control. In some embodiments, the control is a non-cancerous cell.

In some embodiments, the level or activity of the urea cycle enzyme (such as ASS1) expressed in the cancer is lower than that of the surrounding tissue. In some embodiments, the level of ASS1 expressed in the cancer is lower than that of control non-cancer cells. In some embodiments, the control non-transformed cells are cells from the same organ or tissue as the cancer (i.e. non-cancerous liver cells as a control for liver cancer cells). The control cells may or may not be from the same individual as the cancer cells.

In another embodiment, the control level of the urea cycle enzyme (such as ASS1) is the average expression level in samples derived from a population of subjects, e.g., the average expression level of the enzyme in a population of subjects without cancer. In another embodiment, the control level constitutes a range of expression of ASS1 in normal tissue. In another embodiment, baseline abundance refers to a pre-treatment level of the ASS1 in a subject. Control levels of expression of ASS1 and other urea cycle enzymes may be available from publicly available databases. In some embodiments, the control level of enzyme used is for the same organ or tissue as the cancer.

In some embodiments, the tissue sample is a control sample from non-transformed or non-cancerous tissue. In some embodiments, the cancer sample and the control sample are from the same individual. In some embodiments, the cancer sample and the control sample are from different individuals. In some embodiments, the cancer sample and the control sample are from the same organ. In some embodiments, a collection of control samples are used. In some embodiments, the expression or activity level of the urea cycle of a collection of control samples is averaged to find a control value.

In some embodiments, the level of the urea cycle enzyme (such as ASS1) protein expressed in the cancer is 1.5, 2, 3, 4, 5, 6, 7, 8, 9, 10, 15, 20, or 50 fold lower in the cancer than in a control. In some embodiments, the protein level ASS1 of a low ASS1 cancer is reduced by about 5%, 10%, 5%, 20%, 25%, 30%, 35%, 40%, 45%, 50%, 55%, 60%, 65%, 70%, or more compared to a control. In some embodiments, the level of ASS1 protein is reduced and/or eliminated in the cancer but not in the surrounding stromal cells. In some embodiments, the level of ASS1 is determined by immunohistochemistry. In some embodiments, the method comprises selecting an individual for treatment with a GCN2 inhibitor based upon the level of the urea cycle enzyme (such as ASS1) protein in the cancer.

In some embodiments, the level of the urea cycle enzyme (such as ASS1) mRNA expressed in the cancer is 1.5, 2, 3, 4, 5, 6, 7, 8, 9, 10, 15, 20, or 50 fold lower in the cancer than in a control. In some embodiments, the mRNA ASS1 of a low ASS1 cancer is reduced by about 5%, 10%, 5%, 20%, 25%, 30%, 35%, 40%, 45%, 50%, 55%, 60%, 65%, 70%, or more compared to a control. In some embodiments, the level of ASS1 is reduced and/or eliminated in the cancer but not in the surrounding stromal cells. In some embodiments, the method comprises selecting an individual for treatment with a GCN2 inhibitor based upon the level of the urea cycle enzyme (such as ASS1) mRNA in the cancer.

In some embodiments, the activity level of the urea cycle enzyme (such as ASS1) expressed in the cancer is 1.5, 2, 3, 4, 5, 6, 7, 8, 9, 10, 15, 20, or 50 fold lower in the cancer than in a control. In some embodiments, the ASS1 activity of a low ASS1 cancer is reduced by about 5%, 10%, 5%, 20%, 25%, 30%, 35%, 40%, 45%, 50%, 55%, 60%, 65%, 70%, or more compared to a control. In some embodiments, the level of ASS1 is reduced and/or eliminated in the cancer but not in the surrounding stromal cells. In some embodiments, the method comprises selecting an individual for treatment with a GCN2 inhibitor based upon the activity level of the urea cycle enzyme (such as ASS1) in the tumor.

In some embodiments, the activity level of the urea cycle enzyme is determined by measuring the presence of an intermediate the urea cycle. In some embodiments, the activity level of ASS1 is determined by measuring the level of argininosuccinate and/or citrulline. In some embodiments, the level of the urea cycle intermediate is measured using mass spectrometry.

In some embodiments, the level of the urea cycle enzyme (such as ASS1) is measured by isotopic labeling. In some embodiments, the labeling is carried out according to Opladen et al., In vivo monitoring of urea cycle activity with ¹³C-acetate as a tracer of ureagenesis, Mol. Gen. and Metabolism, 117 (1): 19-26 (2016). In some embodiments, subjects are administered ¹³C sodium acetate and the presence of ¹³C in one or more urea cycle intermediates is measured.

In some embodiments, the level of the urea cycle enzyme is measured using a fluorometric or colorimetric assay. In some embodiments, the level of homocitrulline and/or citrulline is measured. In some embodiments, the level of arginine is detected. In some embodiments, the assay comprises an enzyme that converts one or more urea cycle enzyme intermediates to other species, which can then be detected using a probe.

In some embodiments, the method comprises scoring an immunohistochemically stained tumor sample. In some embodiments, cells in the tumor sample are scored as having low, medium, or high levels of ASS1. In some embodiments, the method comprises determining the percentage of cells that have reduced or absent expression of ASS1. In some embodiments, a cancer is determined to be low-ASS1 if less than 90%, less than 80%, less than 70%, less than 60%, less than 50%, less than 40%, less than 30%, less than 20%, or less than 5% of the cells express ASS1. In some embodiments, the method comprises selecting a patient for treatment based upon an immunohistochemistry score.

In some embodiments, the cells in the tumor sample are scored as having low ASS1 if the expression of ASS1 in the tumor cells appears to be relatively lower than that of a control sample. In some embodiments, software is used to score the immunohistochemically stained tumor sample.

In some embodiments immunohistochemically scoring is performed as in Ohsima et al. (Scientific Reports DOI: 10.1038/srep45504 (2017)). Briefly, the expression of ASS1 can be assessed using a visual grading system on the basis of the intensity of staining signals observed using a light microscope. High intensity (score 3), intermediate intensity (score 2), and low intensity (score 1) were defined as strong, medium, and weak staining, respectively. H-scores can be assigned using the following formula: [1×(% cells of score 1)+2×(% cells of score 2)+3×(% cells of score 3)]. H-scores of the tumor invasive front and the tumor center can be obtained by averaging the H-scores of four random fields of each lesion at 200× magnification and then normalized to the whole H-score of the same specimen. The tumor invasive front can be defined as a tumor lesion within 600 μm from the tumor border.

In some embodiments, ASS1 expression or activity may be a reduction in expression or activity of about 5%, 10%, 5%, 20%, 25%, 30%, 35%, 40%, 45%, 50%, 55%, 60%, 65%, 70%, or more, in at least 50% of tumor cells as compared to expression or activity in an appropriate control sample known to be cancer free or containing ASS1-positive cancer cells. In certain embodiments, ASS1 expression or activity is reduced by at least 1.5 fold, or at least 2 fold as compared to expression or activity in a control sample. In some embodiments, a patient is selected for treatment with a GCN2 inhibitor if the ASS1 activity is reduced by at least to fold as compared to expression or activity in the control sample.

In some embodiments the level of ASS1 is reduced compared to the level of the ASS1 in a non-malignant tissue of the same origin as the cancer as measured under identical assay conditions, using e.g., any RNA and or protein detection method suitable for measuring ASS1 levels, including those described herein.

In some embodiments, a portion of the tumor has reduced ASS1 level or activity. In some embodiments, the entire tumor has reduced ASS1 level or activity.

In some embodiments, the administration of the compound, salt, or composition reduces tumor growth, tumor proliferation, or tumorigenicity in the individual. In some embodiments, the compound, salt, or composition may be used in a method of reducing tumor growth, tumor proliferation, or tumorigenicity in an individual in need thereof. In some embodiments, tumor growth is slowed or arrested. In some embodiments, tumor growth is reduced at least about 10%, 20%, 30%, 40%, 50%, 60%, 70%, 80%, or 90%. In some embodiments, the tumor is reduced in size. In some embodiments, tumor size is reduced at least about 10%, 20%, 30%, 40%, 50%, 60%, 70%, 80%, or 90%. In some embodiments, tumor metastasis is prevented or slowed. In some embodiments, the tumor growth, tumor proliferation, or tumorigenicity is compared to the tumor growth, tumor proliferation, or tumorigenicity in the individual prior to the administration of the compound, salt, or composition. In some embodiments, the tumor growth, tumor proliferation, or tumorigenicity is compared to the tumor growth, tumor proliferation, or tumorigenicity in a similar individual or group of individuals. Methods of measuring tumor growth, tumor proliferation, and tumorigenicity are known in the art, for example by repeated imaging of the individual.

In some embodiments, administration of the compound, salt, or composition induces apoptosis of cancer cells. In some embodiments, apoptosis of cancer cells is increased at least 10%, at least 20%, at least 30%, at least 40% or at least 50% upon administration.

GCN2 induces CHOP as part of the amino acid starvation response and PERK as part of the ER stress response. Accordingly, in some embodiments, the administration of the compound, salt, or composition reduces CHOP induction. In some embodiments, the level of CHOP is reduced by at least 10%, at least 20%, at least 30%, at least 40% or at least 50% upon administration of the GCN2 inhibitor. In some embodiments, the level of CHOP is not significantly reduced by the GCN2 inhibitor. In some embodiments, CHOP induction is measured by detecting CHOP protein. In some embodiments, CHOP induction is measured using a reporter system, for example the CHOP promoter or UTR linked to a sequence encoding a fluorescent protein. In some embodiments, CHOP is measured using western blot. In some embodiments CHOP induction is measured by immunohistochemistry.

PERK is a transmembrane kinase located in the ER membrane. Under stress conditions, PERK is released from its binding partner BiP and dimerizes to become an active kinase. In some embodiments, the administration of the compound, salt, or composition decreases the activity of PERK. In some embodiments, PERK activity is reduced by at least 10%, at least 20%, at least 30%, at least 40% or at least 50% upon administration of the GCN2 inhibitor. In some embodiments, PERK activation is assessed by measuring phosphorylation of its substrate, the eukaryotic translation-initiation factor 2α (eIF2α). In some embodiment, phosphorylation of PERK is measured using immunohistochemistry, western blot, or mass spectrometry.

In some embodiments, the administration of the compound, salt, or composition decreases the level of CHOP and decreases the activity of PERK. In some embodiments, the level of CHOP and the activity of PERK are both decreased by at least 10%, at least 20%, at least 30%, at least 40% or at least 50% upon administration of the GCN2 inhibitor.

In some embodiments, the administration of the compound, salt, or composition decreases the level of CHOP but does not significantly affect the activity of PERK. In some embodiments, the level of CHOP is reduced by at least 10%, at least 20%, at least 30%, at least 40% or at least 50% upon administration of the GCN2 inhibitor.

In some embodiments, the administration of the compound, salt, or composition decreases the level of ATF. In some embodiments, the level of ATF is reduced by at least 10%, at least 20%, at least 30%, at least 40% or at least 50% upon administration of the GCN2 inhibitor. In some embodiments, the level of ATF is determined by detecting ATF protein. In some embodiments, the level of ATF is determined using a reporter system, for example the ATF promoter or UTR linked to a sequence encoding a fluorescent protein. In some embodiments, the level of ATF is detected using immunohistochemistry, mass spectrometry or western blot.

Also provided herein is a method of treating a disease in an individual, wherein the disease involves overexpression of GCN2. In some embodiments, provided herein is a method of treating a disease in an individual, wherein the disease involves activation of GCN2. In some embodiments, GCN2 is overexpressed and/or activated in a specific tissue or cell type, such as a cancer cell.

In some embodiments, the methods provided herein inhibit a stress response in a cell. In some embodiments, the stress response is involved protecting cancer cells. In some embodiments, the stress response relates to amino acid starvation. In some embodiments, the stress response is the unfolded protein response. In some embodiments, the stress response is an ER stress response.

In some embodiments, the methods provided herein result in reduced phosphorylation of GCN2. In some embodiments, downstream signaling by GCN2 is reduced. In some embodiments, phosphorylation of eIF2α kinase is reduced.

In some embodiments, the tumor environment has a low level of an amino acid. In some embodiments, the tumor environment has a low level of arginine. In some embodiments, the cancer cell has a low level of arginine.

In some embodiments, the method comprises delivering a second therapeutic agent to the individual. In some embodiments, a compound or salt thereof described herein or a composition described herein may be used in treating cancer in combination with other anticancer agents such as an anti-neoplastic agent, an immune checkpoint inhibitor, or any other suitable anti-cancer agent.

### II. ASS1 Detection Methods

In some embodiments, provided herein is a method of treating a cancer with a low level of a urea cycle protein (such as ASS1) comprising detecting the level of a urea cycle enzyme and administering a GCN2 inhibitor if the cancer has a low level of the urea cycle protein. The level of expression of a urea cycle enzyme (such as ASS1) in a sample obtained from a subject may be assayed by any of a wide variety of techniques and methods, which transform the enzyme within the sample into a moiety that can be detected and/or quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, immunoblotting, Western blotting, Northern blotting, electron microscopy, mass spectrometry, e.g., MALDI-TOF and SELDI-TOF, immunoprecipitation, immunofluorescence, immunocytochemistry, immunohistochemistry, enzyme linked immunosorbent assays (ELISAs), e.g., amplified ELISA, quantitative blood based assays, e.g., serum ELISA, quantitative urine based assays, flow cytometry, Southern hybridizations, array analysis, gel electrophoresis, flow cytometry, methylation specific PCR, nanostring, RNAseq, and the like, and combinations or subcombinations thereof.

In one embodiment, the level of expression of ASS1 is determined by detecting a transcribed polynucleotide, or portion thereof, e.g., mRNA, or cDNA, of the gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays, Northern blotting and in situ hybridization. Other suitable systems for mRNA sample analysis include microarray analysis (e.g., using Affymetrix's microarray system or Illumina's BeadArray Technology).

In one embodiment, the level of expression of the urea cycle enzyme (such as ASS1) is determined using a nucleic acid probe. The term “probe”, as used herein, refers to any molecule that is capable of selectively binding to a specific biomarker and/or is useful for identifying the presence or properties of the biomarker. Probes can be synthesized by one of skill in the art, or derived from appropriate biological preparations. Probes can be specifically designed to be labeled, by addition or incorporation of a label. Examples of molecules that can be utilized as probes include, but are not limited to, RNA, DNA, proteins, antibodies, and organic molecules. As indicated above, isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to the mRNA. The nucleic acid probe can be, for example, a full-length cDNA, or a portion thereof, such as an oligonucleotide of at least about 7, 10, 15, 20, 25, 30, 35, 40, 45, 50, 100, 250 or about 500 nucleotides in length and sufficient to specifically hybridize under appropriate hybridization conditions to the biomarker genomic DNA. In a particular embodiment, the probe will bind the ASS1 genomic DNA under stringent conditions. Such stringent conditions, for example, hybridization in 6× sodium chloride/sodium citrate (SSC) at about 45° C., followed by one or more washes in 0.2× SSC, 0.1% SDS at 50-65° C., are known to those skilled in the art and can be found in Current Protocols in Molecular Biology, Ausubel et al, eds., John Wiley & Sons, Inc. (1995), sections 2, 4, and 6, the teachings of which are hereby incorporated by reference herein. Additional stringent conditions can be found in Molecular Cloning: A Laboratory Manual, Sambrook et al, Cold Spring Harbor Press, Cold Spring Harbor, N.Y. (1989), chapters 7, 9, and 11, the teachings of which are hereby incorporated by reference herein.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface, for example, in an Affymetrix gene chip array, and the probe(s) are contacted with mRNA. A skilled artisan can readily adapt mRNA detection methods for use in determining the level of the biomarker mRNA.

Other known methods for detecting the urea cycle enzyme at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), Immunoelectrophoresis, radioimmunoassay (RIA), immunofluorescent assays, and Western blotting.

Antibodies used in immunoassays to determine the level of expression of the urea cycle enzyme (such as ASS1) may be labeled to a detectable label. The term “labeled”, with regard to the probe or antibody, encompasses direct labeling of the probe or antibody by incorporation of a label (e.g., a radioactive atom), coupling (i.e., physically linking) a detectable substance to the probe or antibody, as well as indirect labeling of the probe or antibody by reactivity with another reagent that is directly labeled.

Examples of indirect labeling include detection of a primary antibody using a fluorescently labeled secondary antibody and end-labeling of a DNA probe with biotin such that it can be detected with fluorescently labeled streptavidin.

In an embodiment, the antibody is labeled, e.g., a radio-labeled, chromophore-labeled, fluorophore-labeled, or enzyme-labeled antibody. In other embodiments, an antibody derivative (e.g., an antibody conjugated with a substrate or with the protein or ligand of a protein-ligand pair, such as biotin-streptavidin), or an antibody fragment (e.g., a single-chain antibody, or an isolated antibody hypervariable domain) which binds specifically with the biomarker is used.

Also provided herein is a method of detecting a low ASS1 cancer comprising incubating a sample with an agent capable of detecting the level of ASS1. In some embodiments, the method comprises contacting a sample with an antibody that binds to ASS1. In some embodiments, the method comprises contacting the sample with a nucleic acid probe for ASS1. In some embodiments, the method comprises comparing the level of ASS1 in a cancer cell sample to a non-cancer cell sample. In some embodiments, the level of ASS1 is measured using immunohistochemistry. In some embodiments, the method comprises administering a GCN2 inhibitor if the cancer is a low ASS1 cancer.

In some embodiments, the level of urea cycle enzyme (such as ASS1) activity or expression is reduced in the cancer. In some embodiments, the level of ASS1 is reduced due to a mutation in the ASS1 gene. In some embodiments the level of ASS1 is reduced due to a mutation in the ASS1 promoter. In some embodiments, the level of ASS1 activity is reduced due to chromatin silencing, such as promoter methylation. In some embodiments the level of ASS1 activity is reduced by an epigenetic mechanism. In certain embodiments, the reduced expression or activity of ASS1 results from methylation of the ASS promoter or inhibition of the ASS promoter. In another embodiment the reduction in expression or activity of ASS results from a DNA mutation (e.g., one or more point mutations, small deletions, insertions, and the like) or a chromosomal abnormality resulting in deletion of the gene. In one embodiment, the cancer is ASS negative, meaning no expression or activity is observed.

In some embodiments, the level of activity of the urea cycle enzyme (such as ASS1) is measured using an enzymatic assay. In some embodiments, the enzymatic assay measures the presence of one or more intermediates in the urea cycle. In some embodiments, the enzymatic assay measures the conversion of citrulline to argininosuccinate or conversion of arginosuccinate into arginine and fumarate.

In some embodiments, the level of mRNA of a urea cycle enzyme (such as ASS1) is reduced. In some embodiments, the level of mRNA is detected by quantitative PCR, FISH, RNAseq, single cell sequencing, or microarray, Northern blot analysis, an RNAase protection assay, digital RNA detection/quantitation.

In some embodiments, the method comprises detecting the level of ASS1 in a sample. In some embodiments, the method comprises detecting the level of ASS1 in a tissue sample. In some embodiments, the tissue sample is a cancer biopsy or resection, blood, or bone marrow.

Immunohistochemistry (IHC) is the demonstration of a cell or tissue constituent in situ by detecting specific antibody/aptamer-antigen interactions where the antibody/aptamer has been tagged with a visible label. The visual marker may be a fluorescent dye, colloidal metal, hapten, radioactive marker, or more commonly an enzyme. IHC protocols are well known in the art; see, e.g., Immunocytochemical Methods and Protocols (second edition), edited by Lorette C. Javois, from Methods in Molecular Medicine, volume 115, Humana Press, 1999 (ISBN 0-89603-570-0).

### III. Methods of Predicting Responsiveness to a GCN2 Inhibitor

Provided herein are method of predicting responsiveness to a GCN2 inhibitor, based upon the surprising finding that cancers that express a low level of a urea cycle enzyme (such as ASS1) are especially responsive to GCN2 inhibitors, compared to cancers that express higher levels of a urea cycle enzyme. In some embodiments, the method comprises detecting the level or activity of a urea cycle enzyme (such as ASS1) in the cancer and administering a GCN2 inhibitor to the individual if the cancer has a low level or activity of the urea cycle enzyme. In some embodiments, the level of the urea cycle enzyme is detected by immunohistochemistry or qPCR. In some embodiments, the method further comprises comparing the level of ASS1 in the cancer to normal tissue. In some embodiments, the GCN2 inhibitor is administered if the level of ASS1 in the cancer is reduced by about 5%, 10%, 5%, 20%, 25%, 30%, 35%, 40%, 45%, 50%, 55%, 60%, 65%, 70%, or more compared to the control.

In some embodiments, provided herein is a method of diagnosing a GCN2 inhibitor-responsive cancer comprising detecting the level of a urea cycle enzyme (such as ASS1) in a cancer. In some embodiments, the method comprises comparing the level or activity of ASS1 in the cancer to the level of ASS1 in control. In some embodiments, the method further comprises administering a GCN2 inhibitor if the level of ASS1 in the cancer is reduced by about 5%, 10%, 5%, 20%, 25%, 30%, 35%, 40%, 45%, 50%, 55%, 60%, 65%, 70%, or more compared to the control. In some embodiments, the level of the urea cycle enzyme is detected using immunohistochemistry or qPCR.

### IV. GCN2 Inhibitors

Any GCN2 inhibitors can be used in accordance with the present invention. In some embodiments, the GCN2 inhibitor is a compound, a pharmaceutically acceptable salt, stereoisomer or tautomer thereof, described in any one of the following U.S. Patents, U.S. Patent Applications and published PCT applications: U.S. Pat. No. 9,073,944 (WO2013/126132), U.S. Pat. No. 9,861,635 (WO2014/180524), U.S. Pat. No. 10,793,563 (WO2019/148136), U.S. Ser. No. 17/639,169 (WO2021/041973), U.S. Ser. No. 17/639,279 (WO2021/041975), U.S. Ser. No. 17/639,183 (WO2021/041970), U.S. Ser. No. 17/639,269 (WO2021/041976), U.S. Ser. No. 17/603,267 (WO2020/210828), U.S. Pat. No. 11,046,699 (WO2019/236631), U.S. Pat. No. 10,696,651 (WO2018/030466), US20210128563 (WO2017220477), and U.S. Ser. No. 17/796,449 (WO2021/165346), each of which is herein incorporated by reference in its entirety and specifically with respect to the GCN2 inhibitors and method of making the GCN2 inhibitors. In some embodiments, the GCN2 inhibitor is an antibody, a nucleic acid, a protein, or a peptide. In some embodiments, the GCN2 inhibitor is a small molecule.

In some embodiments, the GCN2 inhibitor is a compound described in U.S. patent Ser. No. 17/796,449 (WO 2021/165346), which is incorporated herein by reference in its entirety. In some embodiments, the GCN2 inhibitor is a compound of Formula (I):

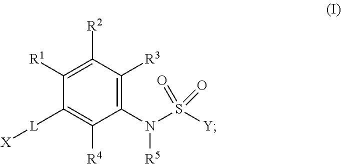

or a pharmaceutically acceptable salt, stereoisomer or tautomer thereof, wherein;
R¹, R², R³ and R⁴ are independently selected from the group consisting of H, halo and C₁₋₃ alkyl optionally substituted with 1-6 fluoro, wherein at least one of R¹, R² R³ and R⁴ is halo or C₁₋₃ alkyl optionally substituted with 1-6 fluoro, or R⁴ is joined to R⁵ to form 5 or 6-membered heterocyclic ring, wherein the 5 or 6-membered heterocyclic ring is optionally substituted with halo and C₁₋₃ alkyl optionally substituted with 1-6 fluoro;
R⁵ is selected from the group consisting of H and C₁₋₃ alkyl optionally substituted with 1-6 fluoro;
L is absent or —C≡C—;
X is a 5, 6, 9 or 10-membered heterocyclic ring comprising 1-4 annular heteroatoms being nitrogen, wherein the 5, 6, 9 or 10-membered heterocyclic ring is substituted with NR⁸R⁹ and optionally further substituted with halo, C₁₋₃ alkyl or NH₂;
R⁸ and R⁹ are independently selected from the group consisting of H, C₁₋₆ alkyl, —C(O) NH₂, —C(O)—C₁₋₆ alkyl, and 5 or 6-membered carbocyclic or heterocyclic, wherein the C₁₋₆ alkyl, —C(O)—C₁₋₆ alkyl, and 5 or 6-membered carbocyclic or heterocyclic are independently optionally substituted with 1-6 substituents selected from the group consisting halo, OH and phenyl,
or R⁸ and R⁹ taken together with the nitrogen form a 6-membered heterocyclic ring;
Y is a 5, 6, 9 or 10-membered carbocyclic or heterocyclic ring; or NH₂, wherein the 5, 6, 9 or 10-membered carbocyclic or heterocyclic ring is optionally substituted with 1-3 substituents selected from the group consisting of halo, OH, CN, —C(O)NR¹³R¹⁴, —NR¹³COR¹⁴, —C(O)OR₁₃, C₁₋₆ alkyl, C₁₋₆ alkoxy, wherein the C₁₋₆ alkyl and C₁₋₆ alkoxy are optionally substituted with 1-6 substituents selected from the group consisting of halo and OH; and
R¹³ and R¹⁴ are independently H or C₁₋₃ alkyl.

In some embodiments, L is —C≡C—.

In some embodiments, X is 6-membered heterocyclic ring comprising 2 annular heteroatoms being nitrogen, wherein the 6-membered heterobicyclic ring is substituted with NH₂.

In some embodiments, a compound is selected from the group consisting of N-(3-((2-Aminopyrimidin-5-yl) ethynyl)-2,4-difluorophenyl)-5-chloro-2-methoxypyridine-3-sulfonamide, 5-Chloro-N-(2,4-difluoro-3-((2-((trans-4-hydroxycyclohexyl)amino)pyrimidin-5-yl) ethynyl)phenyl)-2-methoxypyridine-3-sulfonamide, and 2,5-Dichloro-N-(2,4-difluoro-3-((2-(((2R)-1-hydroxypropan-2-yl)amino)pyrimidin-5-yl) ethynyl)phenyl)-3-(hydroxymethyl)benzenesulfonamide, a pharmaceutically acceptable salt, stereoisomer or tautomer thereof. In some embodiments, the GCN2 inhibitor is

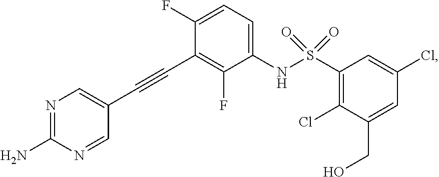 [SMILES: Nc1ncc(C#Cc2c(F)ccc(NS(=O)(=O)c3cc(Cl)cc(CO)c3Cl)c2F)cn1]

a pharmaceutically acceptable salt, stereoisomer or tautomer thereof.

In some embodiments, L is absent and the GCN2 inhibitor is a compound of Formula (I-1):

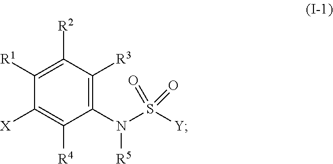

or a pharmaceutically acceptable salt, stereoisomer or tautomer thereof, wherein;
R¹, R², R³ and R⁴ are independently selected from the group consisting of H, halo and C₁₋₃ alkyl optionally substituted with 1-6 fluoro, wherein at least one of R¹, R² R³ and R⁴ is halo or C₁₋₃ alkyl optionally substituted with 1-6 fluoro, or R⁴ is joined to R⁵ to form 5 or 6-membered heterocyclic ring, wherein the 5 or 6-membered heterocyclic ring is optionally substituted with halo and C₁₋₃ alkyl optionally substituted with 1-6 fluoro;
R⁵ is selected from the group consisting of H and C₁₋₃ alkyl optionally substituted with 1-6 fluoro;
X is a 9 or 10-membered fused heterobicyclic ring comprising 1-4 annular heteroatoms being nitrogen, wherein the 9 or 10-membered fused heterobicyclic ring is substituted with NR⁸R⁹ and optionally further substituted with halo, C₁₋₃ alkyl or NH₂;
R⁸ and R⁹ are independently selected from the group consisting of H, C₁₋₆ alkyl, —C(O) NH₂, —C(O)—C₁₋₆ alkyl, and 5 or 6-membered carbocyclic or heterocyclic, wherein the C₁₋₆ alkyl, —C(O)—C₁₋₆ alkyl, and 5 or 6-membered carbocyclic or heterocyclic are independently optionally substituted with 1-6 substituents selected from the group consisting halo, OH and phenyl,
or R⁸ and R⁹ taken together with the nitrogen form a 6-membered heterocyclic ring;
Y is a 5, 6, 9 or 10-membered carbocyclic or heterocyclic ring; or NH₂, wherein the 5, 6, 9 or 10-membered carbocyclic or heterocyclic ring is optionally substituted with 1-3 substituents selected from the group consisting of halo, OH, CN, —C(O)NR¹³R¹⁴, —NR¹³COR¹⁴, —C(O)OR₁₃, C₁₋₆ alkyl, C₁₋₆ alkoxy, wherein the C₁₋₆ alkyl and C₁₋₆ alkoxy are optionally substituted with 1-6 substituents selected from the group consisting of halo and OH; and
R¹³ and R¹⁴ are independently H or C₁₋₃ alkyl.

In some embodiments, the GCN2 inhibitor is a compound of Formula (I-1), a pharmaceutically acceptable salt, stereoisomer or tautomer thereof, provided that when X is a 9-membered fused heterobicyclic ring comprising 1-4 annular heteroatoms being nitrogen, then the 9 membered fused heterobicyclic ring is substituted with NR⁸R⁹ only, and Y is a 6-membered heterocyclic ring comprising 1-4 annular heteroatoms being nitrogen, wherein the 6-membered heterocyclic ring is optionally substituted with 1-3 substituents selected from the group consisting of halo, OH, CN, —C(O)NR¹³R¹⁴, —NR¹³COR¹⁴, —C(O)OR₁₃, C₁₋₆ alkyl, C₁₋₆ alkoxy, wherein the C₁₋₆ alkyl and C₁₋₆ alkoxy are optionally substituted with 1-6 substituents selected from the group consisting of halo and OH; and when X is a 10-membered fused heterobicyclic ring comprising 1-4 annular heteroatoms being nitrogen, wherein the 10 membered fused heterobicyclic ring is substituted with NH₂ only, one of R¹, R², R³ and R⁴ is halo or C₁₋₃ alkyl, the remainder of R¹, R², R³ and R⁴ are H, R⁵ is H, and Y is 5 or 6-membered carbocyclic or heterocyclic ring, then the 5 or 6-membered carbocyclic or heterocyclic ring is substituted with 2 or 3 substituents selected from the group consisting of halo, OH, CN, —C(O)NR¹³R¹⁴, —NR¹³COR¹⁴, —C(O)OR₁₃, C₁₋₆ alkyl, C₁₋₆ alkoxy, wherein the C₁₋₆ alkyl and C₁₋₆ alkoxy are optionally substituted with 1-6 substituents selected from the group consisting of halo and OH.

X can be a 9 or 10-membered fused heterobicyclic ring system comprising 1-4 N annular heteroatoms. X can be a 9 or 10-membered fused heterobicyclic ring system comprising 1 N heteroatom. X can be a 9 or 10-membered fused heterobicyclic ring system comprising 2 N annular heteroatoms. X can be a 9 or 10-membered fused heterobicyclic ring system comprising 3 N annular heteroatoms. X can be a 9 or 10-membered fused heterobicyclic ring system comprising 4 N annular heteroatoms. X can be a 9 or 10-membered fused heterobicyclic ring system comprising 1-4 N annular heteroatoms which is substituted with groups R⁶ and R⁷; wherein one of R⁶ and R⁷ is NR⁸R⁹ and the other is H, NH₂ or halo.

X can be a 9 or 10-membered fused heterobicyclic ring system comprising 1-4 N annular heteroatoms which is substituted with NR⁸R⁹ and optionally further substituted with halo, C₁₋₃ alkyl or NH₂. The 9 or 10-membered fused heterobicyclic ring can be aromatic or non-aromatic. In some embodiments, the 9 or 10-membered fused heterobicyclic ring is aromatic.

X can be a 9 or 10-membered fused heterobicyclic ring which is substituted with groups R⁶ and R⁷, wherein one of R⁶ and R⁷ is NR⁸R⁹ and the other is H, NH₂ or halo. In some embodiments, R⁸ and R⁹ are independently selected from the group consisting of H, C₁₋₆ alkyl, —C(O)NH₂, —C(O)—C₁₋₆ alkyl, and 5 or 6-membered carbocyclic or heterocyclic, wherein the C₁₋₆ alkyl, —C(O)—C₁₋₆ alkyl, and 5 or 6-membered carbocyclic or heterocyclic are independently optionally substituted with 1-6 substituents selected from the group consisting halo, OH and phenyl, or or R⁸ and R⁹ taken together with the nitrogen form a 6-membered heterocyclic ring. In some embodiments, R⁶ and R⁷ are independently selected from the group consisting of: H, NH₂, NHCH₃, N(CH₃)₂, F, NHCH₂CH₂OH, NHCH(CH₃) CH₂OH, NHCH(CH₂OH)₂, NHCH(CH₂OH) (C₆H₅), NHCOCH₃, NHCOCH₂CH₃, NHCOCH(CH₃)₂, NHCOC(CH₃)₃,

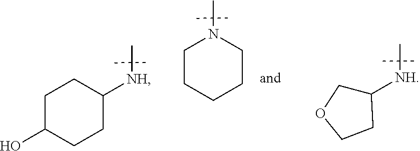

X can be a 9 or 10-membered fused heterobicyclic ring system comprising 1-4 N annular heteroatoms which is substituted with NH₂.

X can be a 10-membered fused heterobicyclic ring system comprising 2 N annular heteroatoms which is substituted with NH₂.

In some embodiments, the heterobicyclic ring system is selected from the group consisting of: quinazoline, quinoline, benzimidazole, isoquinoline, pyrido[2,3-d]pyrimidine, pyrido[3,2-d]pyrimidine, pyrido[2,3-b]pyrazine, pyrido[3,4-b]pyrazine, pteridine, quinoxaline, purine, pyrrolo[2,3-d]pyrimidine, pyrrolo[3,2-d]pyrimidine, indazole and pyrrolo[2,3-b]pyridine; and the optional substituents are the groups R⁶ and R⁷; wherein one of R⁶ and R⁷ is NR⁸R⁹ and the other is H, NH₂ or halo.

In some embodiments, the heterobicyclic ring system is selected from the group consisting of: quinazoline, quinoline, benzimidazole, isoquinoline, pyrido[2,3-d]pyrimidine, pyrido[3,2-d]pyrimidine, pyrido[2,3-b]pyrazine, pyrido[3,4-b]pyrazine, pteridine, quinoxaline, purine, pyrrolo[2,3-d]pyrimidine, pyrrolo[3,2-d]pyrimidine, indazole and pyrrolo[2,3-b]pyridine; and the optional substituents are the groups R⁶ and R⁷, one of R⁶ and R⁷ is NR⁸R⁹ and the other is H, NH₂ or halo.

In some embodiments, the heterobicyclic ring system is selected from the group consisting of: quinazoline, quinoline, benzimidazole, isoquinoline, pyrido[2,3-d]pyrimidine, pyrido[3,2-d]pyrimidine, pyrido[2,3-b]pyrazine, pyrido[3,4-b]pyrazine, pteridine, quinoxaline, purine, pyrrolo[2,3-d]pyrimidine, pyrrolo[3,2-d]pyrimidine, indazole and pyrrolo[2,3-b]pyridine, each of which is substituted with NH₂.

X can be a substituted quinazoline ring system, wherein the substituent is NH₂.

X can be selected from the group consisting of:

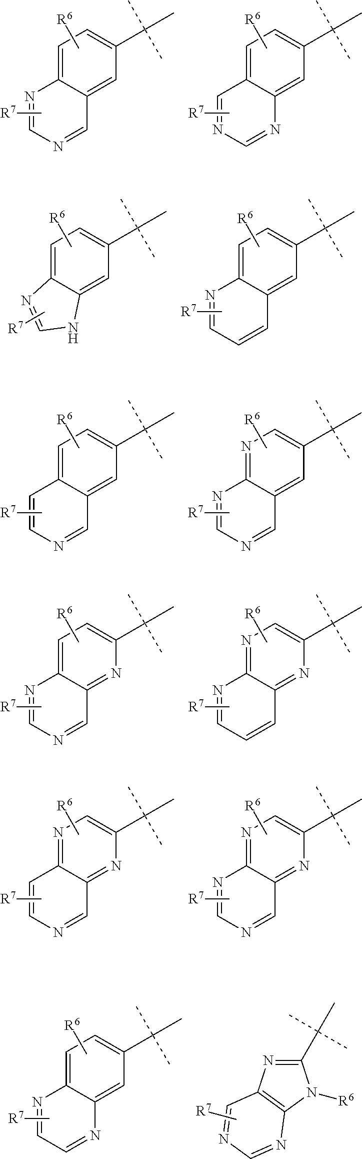

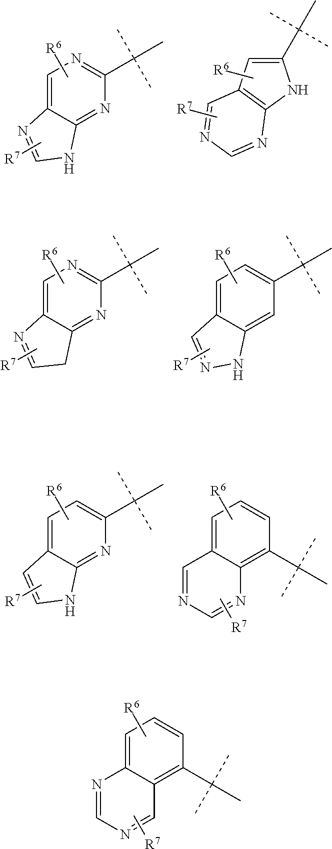

wherein R⁶ and R⁷ may be attached at any available position of the heterobicyclic ring, one of R⁶ and R⁷ is NR⁸R⁹ and the other is H, NH₂ or halo.

X can be:

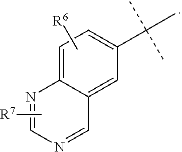

X can be selected from the group consisting of:

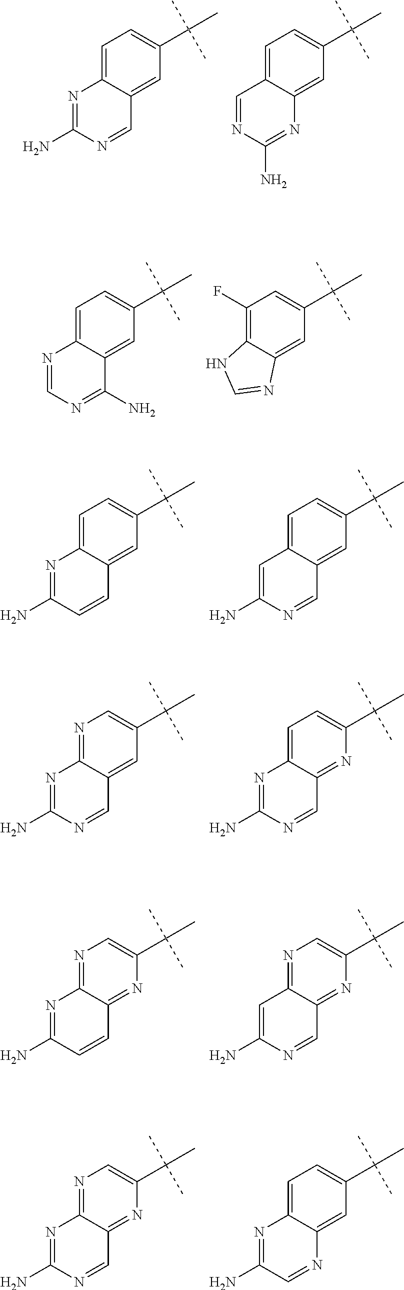

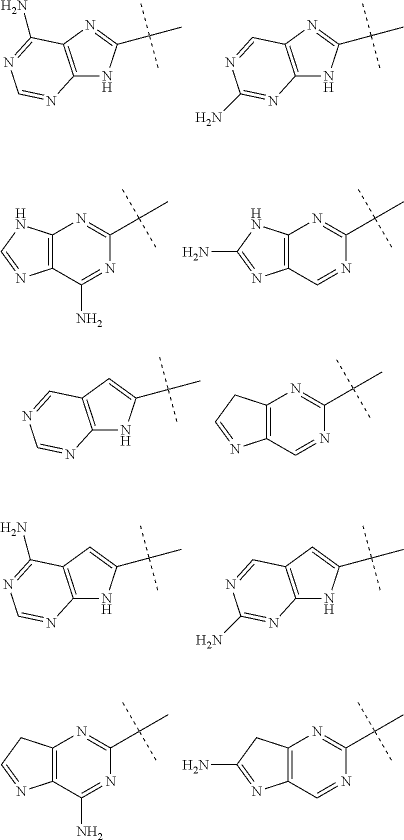

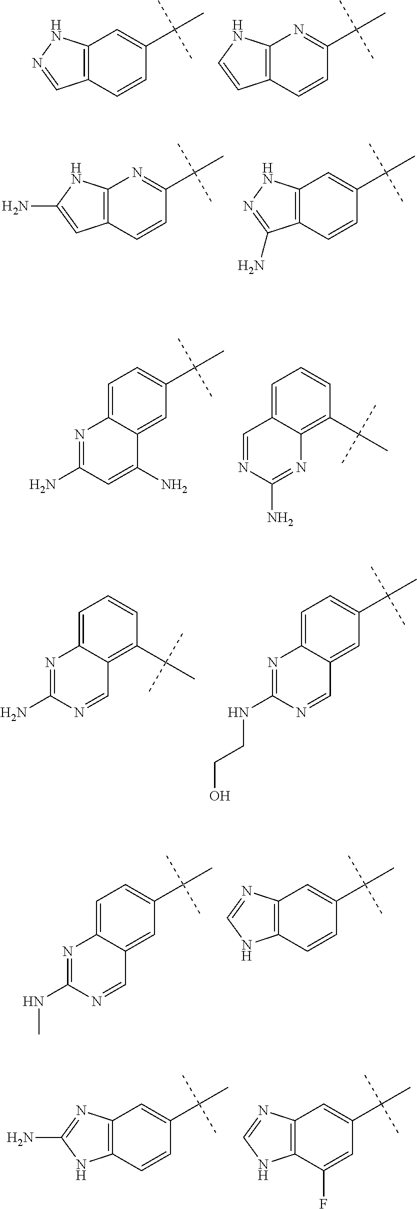

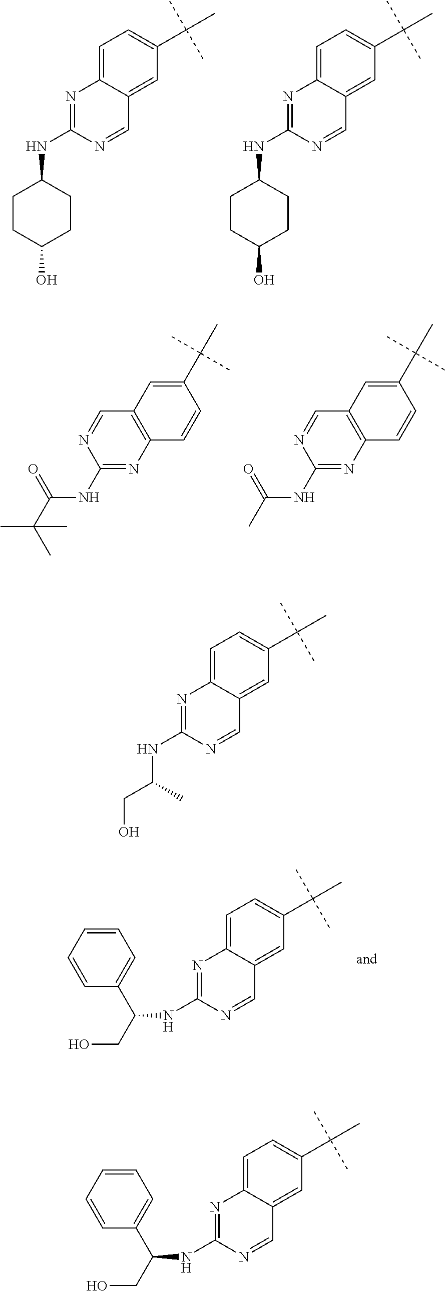

In some embodiments, one of R⁶ and R⁷ is NR⁸R⁹ and the other is H, NH; or halo. R⁶ and R⁷ may be independently selected from: H, NH₂, NHCH₃, N(CH₃)₂, F, NHCH₂CH₂OH, NHCH(CH₃)CH₂OH, NHCH(CH₂OH)₂, NHCH(CH₂OH)(C₆H₅), NHCOCH₃, NHCOCH₂CH₃, NHCOCH(CH₃)₂, NHCOC(CH₃)₃,

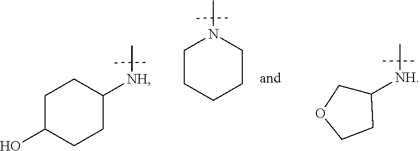

R⁶ can be H and R⁷ can be NH₂.

Y can be selected from the group consisting of:

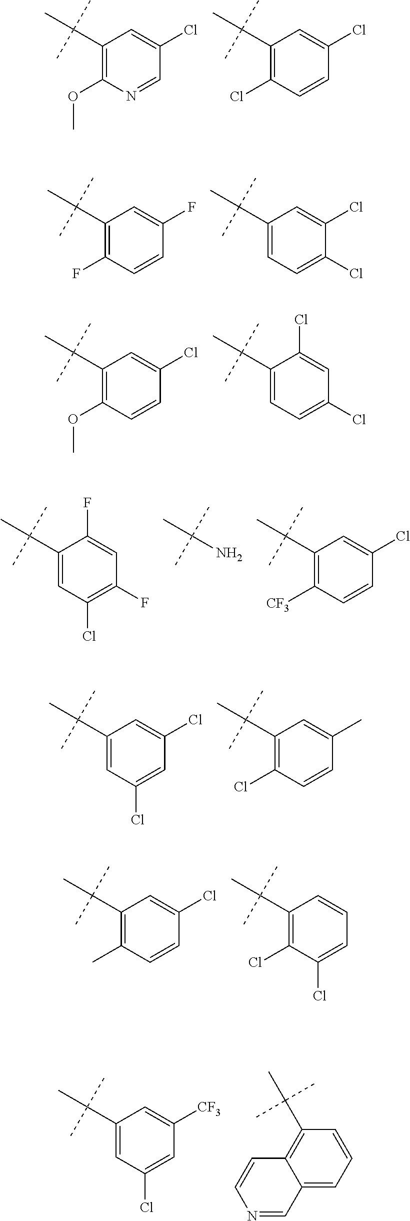

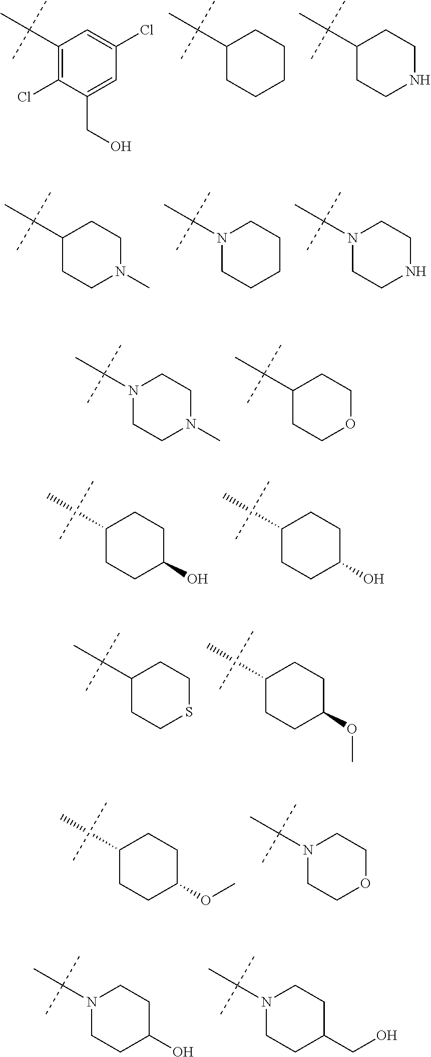

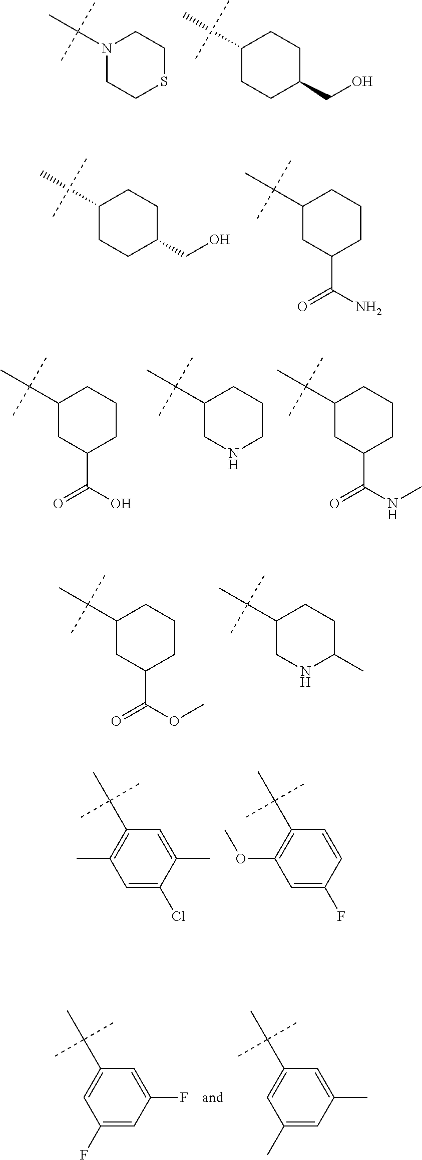

In some embodiments, the GCN2 inhibitor is a compound of formula (1-2):

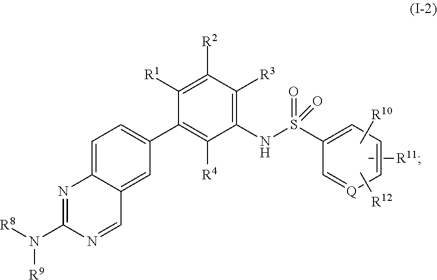

or a pharmaceutically acceptable salt, stereoisomer or tautomer thereof, wherein
Q is N, C or CH, and
R¹⁰, R¹¹ and R¹² are independently selected from the group consisting of: H, halo, OH, CN, —C(O)NR¹³R¹⁴, —NR¹³COR¹⁴, —C(O)OR₁₃, C₁₋₆ alkyl, C₁₋₆ alkoxy, wherein the C₁₋₆ alkyl and C₁₋₆ alkoxy are optionally substituted with 1-6 substituents selected from the group consisting of halo and OH, wherein R¹³ and R¹⁴ are independently H or C₁₋₃ alky.

In some embodiments, R¹ and R⁴ are F, and R² and R³ are H.

The GCN2 inhibitor can be selected from the group consisting of:
N-(3-(2-aminoquinazolin-6-yl)-2,4-difluorophenyl)-5-chloro-2-methoxypyridine-3-sulfonamide;
N-[3-(2-aminoquinazolin-6-yl)-2,4-difluorophenyl]-2,5-dichlorobenzene-1-sulfonamide;
N-{3-[(2-aminopyrimidin-5-yl) ethynyl]-2,4-difluorophenyl}-5-chloro-2-methoxypyridine-3-carboxamide;
N-{3-[6-(2-aminopyrimidin-5-yl)pyridin-3-yl]-2,4-difluorophenyl}-5-chloro-2-methoxypyridine-3-sulfonamide;
N-[3-(2-aminoquinazolin-7-yl)-2,4-difluorophenyl]-2,5-dichlorobenzene-1-sulfonamide;
N-[3-(2-aminoquinazolin-7-yl)-2,4-difluorophenyl]-5-chloro-2-methoxypyridine-3-sulfonamide;
5-({3-[5-(5-chloro-2-methoxypyridin-3-yl)-1,3,4-oxadiazol-2-yl]-2,6-difluorophenyl}ethynyl)pyrimidin-2-amine;
N-[3-(2-aminoquinazolin-6-yl)-2,4-difluorophenyl]-2,5-difluorobenzene-1-sulfonamide;
N-[3-(2-aminoquinazolin-6-yl)-2,4-difluorophenyl]-3,4-dichlorobenzene-1-sulfonamide;
N-[3-(2-aminoquinazolin-6-yl)-2,4-difluorophenyl]-5-chloro-2-methoxybenzene-1-sulfonamide;
N-[3-(2-aminoquinazolin-6-yl)-2-methylphenyl]-2,5-dichlorobenzene-1-sulfonamide;
N-[3-(2-aminoquinazolin-6-yl)-2,6-difluorophenyl]-2,5-dichlorobenzene-1-sulfonamide;
N-[3-(2-aminoquinazolin-6-yl)-2-fluorophenyl]-2,5-dichlorobenzene-1-sulfonamide;
N-[3-(2-aminoquinazolin-6-yl)-5-fluorophenyl]-2,5-dichlorobenzene-1-sulfonamide;
N-[3-(2-aminoquinazolin-6-yl)-2,4-difluorophenyl]-2,4-dichlorobenzene-1-sulfonamide;
N-[3-(2-aminoquinazolin-6-yl)-4-methylphenyl]-2,5-dichlorobenzene-1-sulfonamide;
N-[3-(4-aminoquinazolin-6-yl)-2,4-difluorophenyl]-2,5-dichlorobenzene-1-sulfonamide
2,5-dichloro-N-[2,4-difluoro-3-(7-fluoro-1H-benzimidazol-5-yl)phenyl]benzene-1-sulfonamide;
N-[3-(2-aminoquinazolin-6-yl)-4-fluorophenyl]-5-chloro-2-methoxypyridine-3-sulfonamide;
N-[3-(2-aminoquinolin-6-yl)-2,4-difluorophenyl]-5-chloro-2-methoxypyridine-3-sulfonamide;
N-[3-(3-aminoisoquinolin-7-yl)-2,4-difluorophenyl]-5-chloro-2-methoxypyridine-3-sulfonamide;
N-[3-(2-aminopyrido[2,3-d]pyrimidin-6-yl)-2,4-difluorophenyl]-5-chloro-2-methoxypyridine-3-sulfonamide;
N-[3-(2-aminopyrido[3,2-d]pyrimidin-6-yl)-2,4-difluorophenyl]-5-chloro-2-methoxypyridine-3-sulfonamide;
N-[3-(6-aminopyrido[2,3-b]pyrazin-2-yl)-2,4-difluorophenyl]-5-chloro-2-methoxypyridine-3-sulfonamide;
N-[3-(7-aminopyrido[3,4-b]pyrazin-3-yl)-2,4-difluorophenyl]-5-chloro-2-methoxypyridine-3-sulfonamide;
N-[3-(2-aminopteridin-6-yl)-2,4-difluorophenyl]-5-chloro-2-methoxypyridine-3-sulfonamide;
N-[3-(2-aminoquinoxalin-6-yl)-2,4-difluorophenyl]-5-chloro-2-methoxypyridine-3-sulfonamide;
N-[3-(6-amino-9H-purin-8-yl)-2,4-difluorophenyl]-5-chloro-2-methoxypyridine-3-sulfonamide;
N-[3-(2-amino-9H-purin-8-yl)-2,4-difluorophenyl]-5-chloro-2-methoxypyridine-3-sulfonamide;
N-[3-(6-amino-9H-purin-2-yl)-2,4-difluorophenyl]-5-chloro-2-methoxypyridine-3-sulfonamide;
N-[3-(8-amino-9H-purin-2-yl)-2,4-difluorophenyl]-5-chloro-2-methoxypyridine-3-sulfonamide;
5-chloro-N-[2,4-difluoro-3-(7H-pyrrolo[2,3-d]pyrimidin-6-yl)phenyl]-2-methoxypyridine-3-sulfonamide;
5-chloro-N-[2,4-difluoro-3-(7H-pyrrolo[3,2-d]pyrimidin-2-yl)phenyl]-2-methoxypyridine-3-sulfonamide;
N-[3-(4-amino-7H-pyrrolo[2,3-d]pyrimidin-6-yl)-2,4-difluorophenyl]-5-chloro-2-methoxypyridine-3-sulfonamide;
N-[3-(2-amino-7H-pyrrolo[2,3-d]pyrimidin-6-yl)-2,4-difluorophenyl]-5-chloro-2-methoxypyridine-3-sulfonamide;
N-[3-(4-amino-7H-pyrrolo[3,2-d]pyrimidin-2-yl)-2,4-difluorophenyl]-5-chloro-2-methoxypyridine-3-sulfonamide;
N-[3-(6-amino-7H-pyrrolo[3,2-d]pyrimidin-2-yl)-2,4-difluorophenyl]-5-chloro-2-methoxypyridine-3-sulfonamide;
5-chloro-N-[2,4-difluoro-3-(1H-indazol-6-yl)phenyl]-2-methoxypyridine-3-sulfonamide;
5-chloro-N-[2,4-difluoro-3-(1H-pyrrolo[2,3-b]pyridin-6-yl)phenyl]-2-methoxypyridine-3-sulfonamide;
N-[3-(2-amino-1H-pyrrolo[2,3-b]pyridin-6-yl)-2,4-difluorophenyl]-5-chloro-2-methoxypyridine-3-sulfonamide;
N-[3-(4-aminoquinazolin-6-yl)-2,4-difluorophenyl]-5-chloro-2-methoxypyridine-3-sulfonamide;
N-[3-(3-amino-1H-indazol-6-yl)-2,4-difluorophenyl]-5-chloro-2-methoxypyridine-3-sulfonamide;
5-chloro-N-[3-(2,4-diaminoquinolin-6-yl)-2,4-difluorophenyl]-2-methoxypyridine-3-sulfonamide;
N-[3-(2-aminoquinazolin-8-yl)-2,4-difluorophenyl]-5-chloro-2-methoxypyridine-3-sulfonamide;
N-[3-(2-aminoquinazolin-5-yl)-2,4-difluorophenyl]-5-chloro-2-methoxypyridine-3-sulfonamide;
5-chloro-N-(2,4-difluoro-3-{2-[(2-hydroxyethyl)amino]quinazolin-6-yl}phenyl)-2-methoxypyridine-3-sulfonamide;
5-chloro-N-{2,4-difluoro-3-[2-(methylamino) quinazolin-6-yl]phenyl}-2-methoxypyridine-3-sulfonamide;
N-[3-(1H-benzimidazol-5-yl)-2,4-difluorophenyl]-5-chloro-2-methoxypyridine-3-sulfonamide;
N-[3-(2-amino-1H-benzimidazol-5-yl)-2,4-difluorophenyl]-5-chloro-2-methoxypyridine-3-sulfonamide;
5-chloro-N-[2,4-difluoro-3-(7-fluoro-1H-benzimidazol-5-yl)phenyl]-2-methoxypyridine-3-sulfonamide;
N-[3-(2-aminoquinazolin-6-yl)-2-methylphenyl]-5-chloro-2-methoxypyridine-3-sulfonamide;
N-[3-(2-aminoquinazolin-6-yl)-2-fluorophenyl]-5-chloro-2-methoxypyridine-3-sulfonamide;
N-[3-(2-aminoquinazolin-6-yl)-5-fluorophenyl]-5-chloro-2-methoxypyridine-3-sulfonamide;
N-[3-(2-aminoquinazolin-6-yl)-2,6-difluorophenyl]-5-chloro-2-methoxypyridine-3-sulfonamide;
N-[3-(2-aminoquinazolin-6-yl)-2,4-difluorophenyl]-5-chloro-2,4-difluorobenzene-1-sulfonamide;
N-[3-(2-aminoquinazolin-6-yl)-2,4-difluorophenyl]sulfuric diamide;
N-[3-(2-aminoquinazolin-6-yl)-4-fluorophenyl]-2,5-dichlorobenzene-1-sulfonamide;
N-[3-(2-aminoquinazolin-6-yl)-2,4-difluorophenyl]-5-chloro-2-(trifluoromethyl)benzene-1-sulfonamide;
N-[3-(2-aminoquinazolin-6-yl)-2,4-difluorophenyl]-3,5-dichlorobenzene-1-sulfonamide;
N-[3-(2-aminoquinazolin-6-yl)-2,4-difluorophenyl]-2-chloro-5-methylbenzene-1-sulfonamide;
N-[3-(2-aminoquinazolin-6-yl)-2,4-difluorophenyl]-5-chloro-2-methylbenzene-1-sulfonamide;
N-[3-(2-aminoquinazolin-6-yl)-2,4-difluorophenyl]-2,3-dichlorobenzene-1-sulfonamide;
N-[3-(2-aminoquinazolin-6-yl)-2,4-difluorophenyl]-3-chloro-5-(trifluoromethyl)benzene-1-sulfonamide;
N-[3-(2-aminoquinazolin-6-yl)-2,4-difluorophenyl]isoquinoline-5-sulfonamide;
5-chloro-N-[2,4-difluoro-3-(2-{[(1r,4r)-4-hydroxycyclohexyl]amino}quinazolin-6-yl)phenyl]-2-methoxypyridine-3-sulfonamide;
5-chloro-N-[2,4-difluoro-3-(2-{[(2R)-1-hydroxypropan-2-yl]amino}quinazolin-6-yl)phenyl]-2-methoxypyridine-3-sulfonamide;
2,5-dichloro-N-[2,4-difluoro-3-(2-{[(1r,4r)-4-hydroxycyclohexyl]amino}quinazolin-6-yl)phenyl]benzene-1-sulfonamide;
2,5-dichloro-N-[2,4-difluoro-3-(2-{[(2R)-1-hydroxypropan-2-yl]amino}quinazolin-6-yl)phenyl]benzene-1-sulfonamide;
2,5-dichloro-N-[2,4-difluoro-3-(2-{[(1r,4r)-4-hydroxycyclohexyl]amino}quinazolin-6-yl)phenyl]-3-(hydroxymethyl)benzene-1-sulfonamide;
2,5-dichloro-N-[2,4-difluoro-3-(2-{[(2R)-1-hydroxypropan-2-yl]amino}quinazolin-6-yl)phenyl]-3-(hydroxymethyl)benzene-1-sulfonamide;
6-[1-(5-chloro-2-methoxypyridine-3-sulfonyl)-5-fluoro-1H-indol-4-yl]quinazolin-2-amine;
6-[1-(5-chloro-2-methoxypyridine-3-sulfonyl)-1H-indol-4-yl]quinazolin-2-amine;
6-[1-(5-chloro-2-methoxypyridine-3-sulfonyl)-5-fluoro-2,3-dihydro-1H-indol-4-yl]quinazolin-2-amine;
6-[1-(5-chloro-2-methoxypyridine-3-sulfonyl)-2,3-dihydro-1H-indol-4-yl]quinazolin-2-amine;
6-[1-(2,5-dichlorobenzene-1-sulfonyl)-5-fluoro-1H-indol-4-yl]quinazolin-2-amine;
6-[1-(2,5-dichlorobenzene-1-sulfonyl)-1H-indol-4-yl]quinazolin-2-amine;
{3-[4-(2-aminoquinazolin-6-yl)-5-fluoro-1H-indole-1-sulfonyl]-2,5-dichlorophenyl}methanol;
6-[1-(2,5-dichlorobenzene-1-sulfonyl)-5-fluoro-2,3-dihydro-1H-indol-4-yl]quinazolin-2-amine;
6-[1-(2,5-dichlorobenzene-1-sulfonyl)-2,3-dihydro-1H-indol-4-yl]quinazolin-2-amine;
{3-[4-(2-aminoquinazolin-6-yl)-5-fluoro-2,3-dihydro-1H-indole-1-sulfonyl]-2,5-dichlorophenyl}methanol;
N-[3-(2-aminoquinazolin-6-yl)-2,4-difluorophenyl]cyclohexanesulfonamide;
N-[3-(2-aminoquinazolin-6-yl)-2,4-difluorophenyl]piperidine-4-sulfonamide;
N-[3-(2-aminoquinazolin-6-yl)-2,4-difluorophenyl]-1-methylpiperidine-4-sulfonamide;
N-[3-(2-aminoquinazolin-6-yl)-2,4-difluorophenyl]piperidine-1-sulfonamide;
N-[3-(2-aminoquinazolin-6-yl)-2,4-difluorophenyl]piperazine-1-sulfonamide;
N-[3-(2-aminoquinazolin-6-yl)-2,4-difluorophenyl]-4-methylpiperazine-1-sulfonamide;
N-[3-(2-aminoquinazolin-6-yl)-2,4-difluorophenyl]oxane-4-sulfonamide;
(1r,4r)-N-[3-(2-aminoquinazolin-6-yl)-2,4-difluorophenyl]-4-hydroxycyclohexane-1-sulfonamide;
(1s,4s)-N-[3-(2-aminoquinazolin-6-yl)-2,4-difluorophenyl]-4-hydroxycyclohexane-1-sulfonamide;
N-[3-(2-aminoquinazolin-6-yl)-2,4-difluorophenyl]thiane-4-sulfonamide;
(1r,4r)-N-[3-(2-aminoquinazolin-6-yl)-2,4-difluorophenyl]-4-methoxycyclohexane-1-sulfonamide;
(1s,4s)-N-[3-(2-aminoquinazolin-6-yl)-2,4-difluorophenyl]-4-methoxycyclohexane-1-sulfonamide;
N-[3-(2-aminoquinazolin-6-yl)-2,4-difluorophenyl]morpholine-4-sulfonamide;
N-[3-(2-aminoquinazolin-6-yl)-2,4-difluorophenyl]-4-hydroxypiperidine-1-sulfonamide;
N-[3-(2-aminoquinazolin-6-yl)-2,4-difluorophenyl]-4-(hydroxymethyl)piperidine-1-sulfonamide;
N-[3-(2-aminoquinazolin-6-yl)-2,4-difluorophenyl]thiomorpholine-4-sulfonamide;
(1r,4r)-N-[3-(2-aminoquinazolin-6-yl)-2,4-difluorophenyl]-4-(hydroxymethyl)cyclohexane-1-sulfonamide;
(1s,4s)-N-[3-(2-aminoquinazolin-6-yl)-2,4-difluorophenyl]-4-(hydroxymethyl)cyclohexane-1-sulfonamide;
3-{[3-(2-aminoquinazolin-6-yl)-2,4-difluorophenyl]sulfamoyl}cyclohexane-1-carboxamide;
3-{[3-(2-aminoquinazolin-6-yl)-2,4-difluorophenyl]sulfamoyl}cyclohexane-1-carboxylic acid;
N-[3-(2-aminoquinazolin-6-yl)-2,4-difluorophenyl]piperidine-3-sulfonamide;
3-{[3-(2-aminoquinazolin-6-yl)-2,4-difluorophenyl]sulfamoyl}-N-methylcyclohexane-1-carboxamide;
methyl 3-{[3-(2-aminoquinazolin-6-yl)-2,4-difluorophenyl]sulfamoyl}cyclohexane-1-carboxylate;
N-[3-(2-aminoquinazolin-6-yl)-2,4-difluorophenyl]-6-methylpiperidine-3-sulfonamide;
N-(6-(3-((5-chloro-2-methoxypyridine)-3-sulfonamido)-2,6-difluorophenyl) quinazolin-2-yl)pivalamide;
N-(6-(3((2,5-dichlorophenyl) sulfonamido)2,6-difluorophenyl) quinazolin-2-yl) acetamide;
N-(3-(2-aminoquinolin-6-yl)-2,4-difluorophenyl)-2,5-dichlorobenzenesulfonamide;
N-(3-(2-aminoquinazolin-6-yl)-2,4-difluorophenyl)-2,5-dichloro-3-(hydroxymethyl)benzenesulfonamide;
N-(3-(2-aminoquinazolin-6-yl)-2,4-difluorophenyl)-4-chloro-2,5-dimethylbenzenesulfonamide;
N-(3-(2-aminoquinazolin-6-yl)-2,4-difluorophenyl)-5-fluoro-2-methoxybenzenesulfonamide;
N-(3-(2-aminoquinazolin-5-yl)-2,4-difluorophenyl)-2,5-dichlorobenzenesulfonamide;
N-(3-(2-aminoquinazolin-6-yl)-2,4-difluorophenyl)-3,5-difluorobenzenesulfonamide;
N-(3-(2-aminoquinazolin-6-yl)-2,4-difluorophenyl)-3,5-dimethylbenzenesulfonamide;
N-(5-(3-((5-chloro-2-methoxypyridine)-3-sulfonamido)-2,6-difluorophenyl) quinazolin-2-yl)pivalamide;
N-(3-(2-aminoquinazolin-6-yl)-2,4-difluorophenyl)-3,4-dimethoxybenzenesulfonamide;
N-(3-(2-aminoquinazolin-6-yl)-2,4-difluorophenyl)-3-fluorobenzenesulfonamide;
N-(3-(2-aminoquinazolin-6-yl)-2,4-difluorophenyl)-4-methoxy-3-methylbenzenesulfonamide;
N-(3-(2-aminoquinazolin-6-yl)-2,4-difluorophenyl)-2-methoxy-5-methylbenzenesulfonamide;
N-(3-(2-aminoquinazolin-6-yl)-2,4-difluorophenyl)-2,5-dimethoxybenzenesulfonamide;
N-(3-(2-aminoquinazolin-6-yl)-2,4-difluorophenyl)-5-ethyl-2-methoxybenzenesulfonamide;
N-(3-(2-aminoquinazolin-6-yl)-2,4-difluorophenyl)-2,5-bis(trifluoromethyl)benzenesulfonamide;
N-(3-(2-aminoquinazolin-6-yl)-2,4-difluorophenyl)benzofuran-5-sulfonamide;
N-(3-(2-aminoquinazolin-6-yl)-2,4-difluorophenyl)-3-fluoro-5-(trifluoromethyl)benzenesulfonamide;
N-(3-(2-aminoquinazolin-6-yl)-2,4-difluorophenyl)-2,3-difluorobenzenesulfonamide;
N-(3-(2-aminoquinazolin-6-yl)-2,4-difluorophenyl)-3-methylbenzenesulfonamide;
2,5-dichloro-N-(3,5-difluoro-4-(2-((2-hydroxyethyl)amino) quinazolin-6-yl)pyridin-2-yl)benzenesulfonamide;
5-chloro-N-(2,4-difluoro-3-(2-(((1s,4s)-4-hydroxycyclohexyl)amino) quinazolin-6-yl)phenyl)-2-methoxypyridine-3-sulfonamide;
N-(3-(2-aminoquinazolin-6-yl)-2,4-difluorophenyl)pyridine-3-sulfonamide;
N-(3-(2-aminoquinazolin-6-yl)-2,4-dichlorophenyl)-5-chloro-2-methoxypyridine-3-sulfonamide;
N-(3-(2-aminoquinazolin-6-yl)-2,4-difluorophenyl)-3,4-difluorobenzenesulfonamide;
N-(3-(2-aminoquinazolin-6-yl)-2,4-difluorophenyl)-5-fluoro-2-methylbenzenesulfonamide;
2,5-dichloro-N-(2,4-difluoro-3-(2-((2-hydroxyethyl)amino) quinazolin-6-yl)phenyl)-3-(hydroxymethyl)benzenesulfonamide;
N-(3-(2-aminoquinazolin-6-yl)-2,4-difluorophenyl)-2-fluoro-5-(trifluoromethyl)benzenesulfonamide;
N-(3-(2-aminoquinazolin-6-yl)-2,4-difluorophenyl)-4-fluoro-3-(trifluoromethyl)benzenesulfonamide;
N-(3-(2-aminoquinazolin-6-yl)-2,4-difluorophenyl)-4-chloro-2-(trifluoromethyl)benzenesulfonamide;
N-(3-(2-aminoquinazolin-6-yl)-2,4-difluorophenyl) quinoxaline-5-sulfonamide;
N-(3-(2-aminoquinazolin-6-yl)-2,4-difluorophenyl)-3-(trifluoromethoxy)benzenesulfonamide;
N-(3-(2-aminoquinazolin-6-yl)-2,4-difluorophenyl)thiophene-3-sulfonamide;
N-(3-(2-aminoquinazolin-6-yl)-2,4-difluorophenyl)-3-isopropylbenzenesulfonamide;
N-(3-(2-aminoquinazolin-6-yl)-2,4-difluorophenyl)thiophene-2-sulfonamide;
N-(3-(2-aminoquinazolin-6-yl)-2,4-difluorophenyl)-4-fluoro-3-methoxybenzenesulfonamide;
N-(3-(2-aminoquinazolin-6-yl)-2,4-difluorophenyl)-3,5-bis(trifluoromethyl)benzenesulfonamide;
N-(3-(2-aminoquinazolin-6-yl)-2,4-difluorophenyl)-3-(trifluoromethyl)benzenesulfonamide;
N-(3-(2-aminoquinazolin-6-yl)-2,4-difluorophenyl)-4-chloro-3-fluorobenzenesulfonamide;
N-(3-(2-aminoquinazolin-6-yl)-2,4-difluorophenyl)-3-chloro-4-methylbenzenesulfonamide;
N-(3-(2-aminoquinazolin-6-yl)-2,4-difluorophenyl)-3-chloro-4-methoxybenzenesulfonamide;
N-(3-(2-aminoquinazolin-6-yl)-2,4-difluorophenyl)-4-chloro-3-(trifluoromethyl)benzenesulfonamide;
N-(3-(2-aminoquinazolin-6-yl)-2,4-difluorophenyl)-2,4,5-trichlorobenzenesulfonamide;
5-chloro-N-(3-(2-((1,3-dihydroxypropan-2-yl)amino) quinazolin-6-yl)-2,4-difluorophenyl)-2-methoxypyridine-3-sulfonamide;
(R)-5-chloro-N-(2,4-difluoro-3-(2-((2-hydroxy-1-phenylethyl)amino) quinazolin-6-yl)phenyl)-2-methoxypyridine-3-sulfonamide;
5-chloro-N-(3-(2-(dimethylamino) quinazolin-6-yl)-2,4-difluorophenyl)-2-methoxypyridine-3-sulfonamide;
5-chloro-N-(2,4-difluoro-3-(2-(piperidin-1-yl) quinazolin-6-yl)phenyl)-2-methoxypyridine-3-sulfonamide;
(S)-5-chloro-N-(2,4-difluoro-3-(2-((1-hydroxypropan-2-yl)amino) quinazolin-6-yl)phenyl)-2-methoxypyridine-3-sulfonamide;
N-(3-(2-aminoquinazolin-6-yl)-2,4-difluorophenyl)-3-chloro-4-fluorobenzenesulfonamide;
N-(3-(2-aminoquinazolin-6-yl)-2,4-difluorophenyl)-3-fluoro-4-methylbenzenesulfonamide;
N-(3-(2-aminoquinazolin-6-yl)-2,4-difluorophenyl)-4-fluoro-3-methylbenzenesulfonamide;
N-(3-(2-aminoquinazolin-6-yl)-2,4-difluorophenyl)-2,3-dihydro-1H-indene-5-sulfonamide;
2,5-dichloro-N-(2,4-difluoro-3-(2-(methylamino) quinazolin-6-yl)phenyl)-3-(hydroxymethyl)benzenesulfonamide;
(S)-5-chloro-N-(2,4-difluoro-3-(2-((2-hydroxy-1-phenylethyl)amino) quinazolin-6-yl)phenyl)-2-methoxypyridine-3-sulfonamide;
5-chloro-N-(2,4-difluoro-3-(2-(((1r,4r)-4-hydroxycyclohexyl)amino) quinazolin-6-yl)phenyl)-2-(trifluoromethyl)benzenesulfonamide;
5-chloro-N-(2,4-difluoro-3-(2-(((1s,4s)-4-hydroxycyclohexyl)amino) quinazolin-6-yl)phenyl)-2-(trifluoromethyl)benzenesulfonamide;
(S)-5-chloro-N-(2,4-difluoro-3-(2-((2-hydroxy-1-phenylethyl)amino) quinazolin-6-yl)phenyl)-2-(trifluoromethyl)benzenesulfonamide;
(R)-5-chloro-N-(2,4-difluoro-3-(2-((2-hydroxy-1-phenylethyl)amino) quinazolin-6-yl)phenyl)-2-(trifluoromethyl)benzenesulfonamide; and
2,5-dichloro-N-(2,4-difluoro-3-(2-((tetrahydrofuran-3-yl)amino) quinazolin-6-yl)phenyl)benzenesulfonamide,
   or a pharmaceutically acceptable salt, stereoisomer or tautomer thereof.

In some embodiments, the GCN2 inhibitor is a compound described in U.S. Pat. No. 11,046,699 (WO2019/236631), which is incorporated herein by reference in its entirety. In some embodiments, the GCN2 inhibitor is a compound of Formula (II):

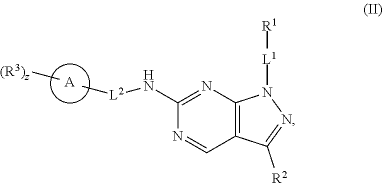

or a pharmaceutically acceptable salt, stereoisomer or tautomer thereof, wherein:
z is an integer from 0 to 6;
ring A is substituted or unsubstituted cycloalkyl, or substituted or unsubstituted heterocycloalkyl;
L¹ and L² are independently a bond, substituted or unsubstituted alkylene, or substituted or unsubstituted heteroalkylene;
R¹ is substituted or unsubstituted aryl or substituted or unsubstituted heteroaryl;
R² is independently hydrogen, halogen, —CX²₃, —CHX²₂, —CH₂X², —OCX²₃, —OCHX²₂, —OCH₂X², —CN, —S(O)₂R^{2A}, —SR^{2A}, —S(O)R^{2A}, —SO₂NR^{2A}R^{2B}, —NHC(O)NR^{2A}R^{2B}, —N(O)₂, —NR^{2A}R^{2B}, —NHNR^{2A}R^{2B}, —C(O)R^{2A}, —C(O)—OR^{2A}, —C(O)NR^{2A}R^{2B}, —C(O)NHNR^{2A}R^{2B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX—CHX³₂, —CH₂X³, —OCX³₃, —OCHX³₂, —OCH₂X³, —CN, —S(O)₂R^{3A}, —SR^{3A}, —S(O)R^{3A}, —SO₂NR^{3A}R^{3B}, —NHC(O)NR^{3A}R^{3B}, —N(O)₂, —NR^{3A}R^{3B}, —NHNR^{3A}R^{3B}, —C(O)R^{3A}, —C(O)—OR^{3A}, —C(O)NR^{3A}R^{3B}, —P(O)R^{3A}R^{3B}, —C(O)NHNR^{3A}R^{3B}, OR^{3A}, —NR^{3A}SO₂R^{3B}, —NR^{3A}C(O)R^{3B}, —NR^{3A}C(O)OR^{3B}, —NR^{3A}OR^{3B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ may optionally be joined to form a substituted or unsubstituted heterocycloalkyl or substituted or unsubstituted heteroaryl;
R^{2A}, R^{2B}, R^{3A}, and R^{3B} are independently hydrogen, —F, —Cl, Br, —I, —CF₃, —CHF₂, —CH₂F, —CCb, —CHCb, —CH₂Cl, —CBr₃, —CHBr₂, —CH₂Br, —CI₃, —CHI₂, —CH₂I, —OCF₃, −OCCb, −OCBrs, —OCI₃, —OCHF₂, −OCHCh, —OCHBr₂, —OCHI₂, —OCH₂F, —OCH₂Cl, —OCH₂Br, —OCH₂I, −Ns, —CN, —SH, −SC¾, —SO₂H, —SO₂CH₃, —SO₂NH₂, —SO₂NHCH₃, —NHC(O)NH₂, —NHC(O)NHCH₃, —NO₂, —NH₂, —NHCHs—C(O)H, —C(O)CH₃, —C(O)OH, —C(O)OCH₃, —C(O)NH₂, —C(O)NHCH₃, —OH, —OCH₃, —NHSO2H, —NHSO₂CH₃, —NHC(O)H, —NCH₃C(O)H, —NHC(O)OH, —NCH₃C(O)OH, —NHOH, —NCH3OH, —NCH3OCH3, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; R^{2A} and R^{2B} may optionally be joined to form a substituted or unsubstituted heterocycloalkyl or substituted or unsubstituted heteroaryl; R^{3A} and R^{3B} may optionally be joined to form a substituted or unsubstituted heterocycloalkyl or substituted or unsubstituted heteroaryl; and X² and X³ are independently halogen;
   with the proviso that when ring A is cyclohexyl, then R³ is not an ortho-substituted-NH₂ or—HNC═(O)t-BuO, or para-substituted —NHSO₂CH₂CH₂CF₃,
   —NHSO₂CH₃, or —OH.

In some such embodiments, the GCN2 inhibitor of formula (II) is of the structure:

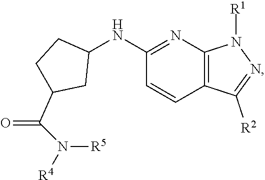

or a pharmaceutically acceptable salt, stereoisomer or tautomer thereof, wherein R¹ and R² are as provided for formula (II), and R⁴ and R⁵ are independently hydrogen or substituted or unsubstituted C₁-C₄ alkyl. For example, in some embodiments, the GCN2 inhibitor is

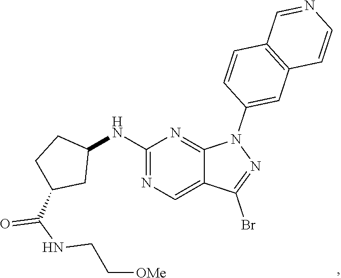

or a pharmaceutically acceptable salt, stereoisomer or tautomer thereof.

In some embodiments, the GCN2 inhibitor of formula (II) is administered in combination with one or more of checkpoint inhibitors. In some embodiments, the GCN2 inhibitor of formula (II) is administered in combination with an anti-PD-1 antibody or an anti-PD-L1 antibodies. In some embodiments, the GCN2 inhibitor of formula (II) is administered in combination with a VEGFR kinase inhibitors, or amino acid depleting enzymes (e.g., Asparaginase).

In some embodiments, the GCN2 inhibitor is a compound described in U.S. Pat. No. 10,793,563 (WO2019/148136), which is incorporated herein by reference in its entirety. In some embodiments, the GCN2 inhibitor is a compound of Formula (III):

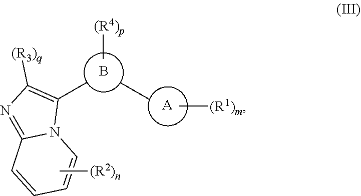

or a pharmaceutically acceptable salt, stereoisomer or tautomer thereof, wherein:

Ring A is selected from a 3-8 membered saturated or partially unsaturated monocyclic carbocyclic ring, phenyl, an 8-10 membered bicyclic aromatic carbocyclic ring, a 4-8 membered partially unsaturated monocyclic heterocyclic ring having 1-2 heteroatoms independently selected from nitrogen, oxygen, or sulfur optionally fused to a 5-6 membered aromatic ring having 0-4 heteroatoms independently selected from nitrogen, oxygen, or sulfur, a 7-12 membered partially unsaturated spirocyclic heterocyclic ring having 1-2 heteroatoms independently selected from nitrogen, oxygen, or sulfur, a 7-12 membered partially unsaturated bicyclic heterocyclic ring having 1-4 heteroatoms independently selected from nitrogen, oxygen, or sulfur, a 7-12 membered partially unsaturated bridged bicyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, an 8-10 membered bicyclic heteroaromatic ring having 1-5 heteroatoms independently selected from nitrogen, oxygen, or sulfur, or

Het, wherein Het is a 4-8 membered saturated monocyclic heterocyclic ring having 1-2 heteroatoms independently selected from nitrogen, oxygen, or sulfur, a 7-12 membered saturated spirocyclic heterocyclic ring having 1-4 heteroatoms independently selected from nitrogen, oxygen, or sulfur, a 7-12 membered saturated bicyclic heterocyclic ring having 1-3 heteroatoms independently selected from nitrogen, oxygen, or sulfur, or a 7-12 membered saturated bridged bicyclic heterocyclic ring having 1-2 heteroatoms independently selected from nitrogen, oxygen, or sulfur;

### Ring B is

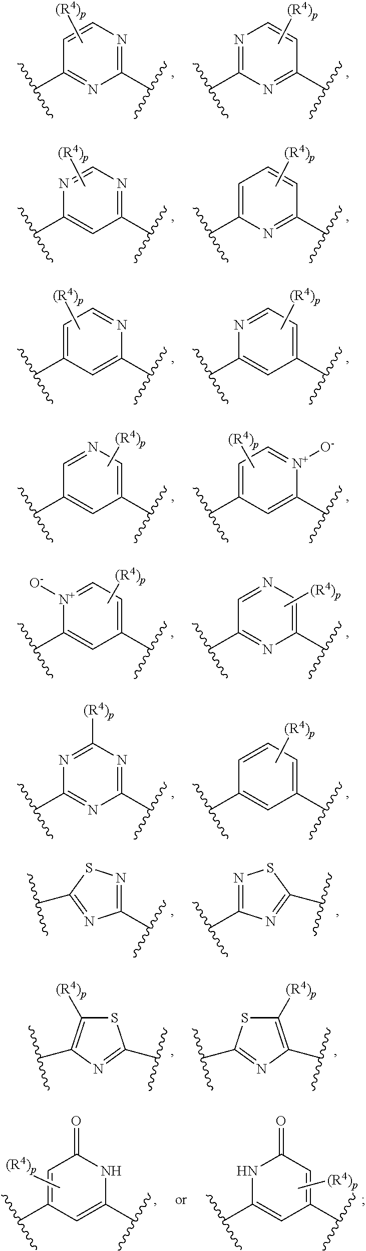

each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or two R groups are optionally taken together to form a bivalent C2-4 alkylene chain;
two R groups are optionally taken together with their intervening atoms to form an optionally substituted 3-7 membered saturated or partially unsaturated monocyclic ring having 0-4 heteroatoms independently selected from nitrogen, oxygen or sulfur;
each R′ is independently hydrogen or a C₁₋₃ aliphatic group optionally substituted with halogen; each of R¹ is independently hydrogen, halogen, —CN, —NO₂, —C(O)R, —C(O)OR, —C(O)NR₂, —C(O)NRS(O)₂R, —C(O)N═S(O)R₂, —NR₂, —NRC(O)R, —NRC(O)NR₂, —NRC(O)OR, —NRS(O)2R, —NRS(O)2NR2, —OR, —ON(R)SO2R, —P(O)R2, —SR, —S(O)R, —S(O)2R, —S(O)(NH)R, —S(O)2N(R)2, —S(NH2)2 (O)OH, —N═S(O)R2, —CH3, —CH2OH, —CH₂NHSO₂CH₃, −CD₃, −CD₂NRS(O)₂R, or R; or:
two R¹ groups are optionally taken together to form ═O or ═NH; or
two R¹ groups are optionally taken together to form a bivalent C₂₋₄ alkylene chain;
each of R² is independently hydrogen, halogen, —CN, —C(O)N(R′)₂, —OR′, —N(R′)₂, —S(O)₂R, —S(O)2N(R)2, —O-phenyl, or an optionally substituted group selected from C₁₋₃ aliphatic, phenyl, 5-6 membered monocyclic heteroaromatic ring having 1-4 heteroatoms independently selected from nitrogen, oxygen, or sulfur, or 4-8 membered saturated monocyclic heterocycle having 1-4 heteroatoms independently selected from nitrogen, oxygen, or sulfur;
R³ is hydrogen, halogen, —CN, —OR′, —N(R′)2, or an optionally substituted group selected from C₁₋₃ aliphatic, phenyl, or a 5-6 membered monocyclic heteroaromatic ring having 1-4 heteroatoms independently selected from nitrogen, oxygen, or sulfur;
R⁴ is hydrogen, halogen, —CN, —OR, —N═S(O)R2, —N(R)2, or an optionally substituted group selected from C₁₋₃ aliphatic, a 4-8 membered saturated or partially unsaturated monocyclic heterocyclic ring having 1-2 heteroatoms independently selected from nitrogen, oxygen, or sulfur, or a 7-12 membered saturated or partially unsaturated spirocyclic heterocyclic ring having 1-2 heteroatoms independently selected from nitrogen, oxygen, or sulfur;
m is 0, 1, 2, 3, 4 or 5;
n is 0, 1, or 2;
p is 0 or 1; and
q is 0 or 1.

In some such embodiments, the GCN2 inhibitor is

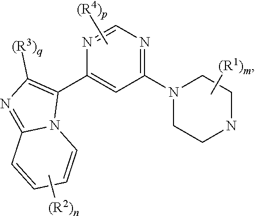

or a pharmaceutically acceptable salt, stereoisomer or tautomer thereof.

In some embodiments, the GCN2 inhibitor is a compound described in U.S. Pat. No. 9,073,944 (WO2013/126132), which is incorporated herein by reference in its entirety. In some embodiments, the GCN2 inhibitor is a compound of Formula (IV):

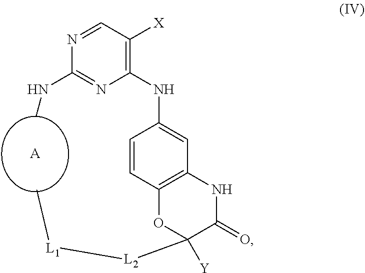

or a pharmaceutically acceptable salt, stereoisomer or tautomer thereof, wherein:

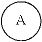

denotes phenylen or 2,3-dihydro-indol-1,6-diyl, each of which is unsubstituted or monosubstituted by OA,
X denotes Hal,
Y denotes alkyl having 1, 2, 3 or 4 C atoms,
L₁ denotes (CH₂)ₙNR¹CO, (CH₂)ₙ, NH(CH₂)ₙ, OCH₂CHOH, NHCO(CH₂)ₙ, CO(CH₂)ₙNR¹, CONR², (CH₂)ₙ, CONR¹, O(CH₂)ₚCONR¹, NR¹CONR³CHR⁴CONR¹, SO₂NR¹ (CH₂)ₚCONR¹ or O(CH₂)ₚNR¹CO,
L₂ denotes O(CH₂)ₚ, (CH₂)ₙNR¹CO, O(CH₂)ₚNR¹CO, CHR⁵NR¹CO or CHR³NR⁴CO,
R¹ denotes H or methyl,
R² denotes denotes piperidinyl, piperazinyl, pyrrolidinyl, morpholinyl, 2,3-dihydro-pyrazolyl, 1,2-dihydro-pyridyl or tetrahydropyranyl, each of which is unsubstituted or mono- or disubstituted by A and/or =0,
R³ and R⁴ together denote an alkylene chain having 2, 3 or 4 CH₂ groups,
R^{b} denotes A or benzyl, A denotes unbranched or branched alkyl having 1-10 C atoms, in which 1-7 H atoms may be replaced by F and/or in which one or two non-adjacent CH and/or CH₂ groups may be replaced by O or N,
Hal denotes F, Cl, Br or I,
n denotes 0, 1, 2, 3 or 4,
p denotes 1, 2, 3 or 4.

In some embodiments, the GCN2 inhibitor is

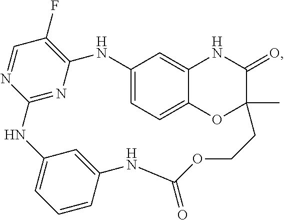 [SMILES: CC12CCOC(=O)Nc3cccc(c3)Nc3ncc(F)c(n3)Nc3ccc(c(c3)NC1=O)O2]

or a pharmaceutically acceptable salt, stereoisomer or tautomer thereof.

In some embodiments, the GCN2 inhibitor is

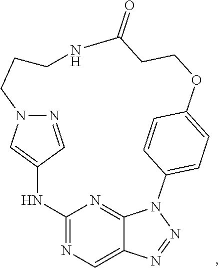 [SMILES: O=C1CCOc2ccc(cc2)-n2nnc3cnc(nc32)Nc2cnn(c2)CCCN1]

or a pharmaceutically acceptable salt, stereoisomer or tautomer thereof.

In some embodiments, the GCN2 inhibitor is a compound described in U.S. Patent No. U.S. Ser. No. 17/603,267 (WO2020/210828), which is incorporated herein by reference in its entirety. In some embodiments, the GCN2 inhibitor is a compound of Formula (V):

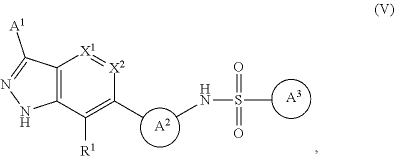

a pharmaceutically acceptable salt, stereoisomer or tautomer thereof, wherein:
X¹ and X² are independently C(R²) or N, wherein X¹ is N and X² is C(R²), X¹ is C(R²) and X² is N, or both X¹ and X² are C(R²);
R¹ is halogen, hydrogen, C₁₋₄ alkyl, C₁₋₄ fluoroalkyl, or cyano;
R² represents independently for each occurrence hydrogen, halogen, C₁₋₄ alkyl, C₁₋₄ fluoroalkyl, cyano, C₁₋₄ alkoxyl, or hydroxyl;
R³ and R⁴ each represent independently for each occurrence hydrogen, C₁₋₄ alkyl, or C₃-7 cycloalkyl; or an occurrence of R³ and R⁴ attached to the same nitrogen atom are taken together with the nitrogen atom to which they are attached to form a 3-7 membered carbocyclyl or heterocyclyl;
R⁵ represents independently for each occurrence hydrogen, C₁₋₄ alkyl, or hydroxyl;
R⁶ represents independently for each occurrence hydrogen, C₁₋₆ alkyl, C₁₋₆ hydroxyalkyl, C₃₋₆ cycloalkyl, 4-7 membered heterocyclyl, 6-10 membered aryl, —(C₁₋₆ alkylene)-N(R³)(R⁴), —(C₁₋₆ alkylene)-N(R³)—C(O)(R⁴), —(C₁₋₆ alkylene)-(5-10 membered heteroaryl), —(C₁₋₆ alkylene)-C₃₋₆ cycloalkyl), —(C₁₋₆ alkylene)-(5-10 membered heterocycloalkyl), —(C₁₋₆ alkylene)-CO₂R³, —(C₁₋₆ alkylene)-C(O)N(R³)(R⁴), —(C₁₋₆ alkylene)-S(O)₂—(C₁₋₆ alkyl), —(C₁₋₆ alkylene)-O—(C₁₋₆ alkyl), or —(C₁₋₆ alkylene)-CN, wherein the C₁₋₆ alkyl, C₃₋₆ cycloalkyl, —(C₁₋₆ alkylene)-(C₃₋₆ cycloalkyl), 4-7 membered heterocyclyl, and —(C₁₋₆ alkylene)-(5-10 membered heterocycloalkyl) may be optionally substituted with 1, 2, or 3 substituents independently selected from the group consisting cyano, halogen, hydroxyl, oxo, and NH₂, and wherein if the 4-7 membered heterocyclyl and —(C₁₋₆ alkylene)-(5-10 membered heterocycloalkyl) contain a suitable ring nitrogen atom, that ring nitrogen may be optionally substituted by C₁₋₃ alkyl or—C(O)—C₁₋₃ alkyl;
R⁷ is C₁-4 alkyl, C₃₋₇ cycloalkyl, or —(C₁₋₆ alkylene)-(C₃₋₇ cycloalkyl);
A¹ is one of the following: x 5-10 membered heterocyclyl or 6-10 membered aryl, each of which is optionally substituted with 1, 2, or 3 substituents independently selected from the group consisting of halogen, C₁₋₆ alkyl, C₁₋₆ hydroxyalkyl, C₁₋₄ haloalkyl, C₃₋₅ cycloalkyl, cyano, hydroxyl, C₁₋₄ alkoxyl, oxo, —N(R³)(R⁴), —(C₁₋₆ alkylene)-N(R³)(R⁴), —C(O)N(R⁵)(R⁶), and —(C₁₋₆ alkylene)-C(O)N(R⁵)(R⁶); or x —C(O)N(R⁵)(R⁶) or —N(R⁵) C(O)(R⁷);
A² is phenylene or a 5-6 membered heteroarylene, each of which is optionally substituted with 1, 2, or 3 substituents independently selected from the group consisting of halogen, C₁₋₄ alkyl, C₁₋₄ haloalkyl, cyano, C₁₋₄ alkoxyl, C₃₋₅ cycloalkyl, and C₃₋₅ halocycloalkyl; and
A³ is phenyl, —CH₂—(C₃₋₆ cycloalkyl), 7-10 membered bicyclic carbocyclyl, or 5-10 membered heterocyclyl, each of which is optionally substituted with 1, 2, or 3 substituents independently selected from the group consisting of halogen, C₁₋₆ alkyl, C₁₋₆ hydroxyalkyl, C₁₋₄ haloalkyl, C₁₋₄ hydroxyfluoroalkyl, C₃₋₅ cycloalkyl, cyano, hydroxyl, C₁₋₄ alkoxyl, C₁₋₄ fluoroalkoxyl, —N(R³)(R⁴), —N(R³)—C(O)(R⁴), —(C₁₋₆ alkylene)-N(R³)(R⁴), —CO₂H, —CO₂ (C₁₋₆ alkyl), —S—(C₁₋₆ alkyl), and —S—(C₁₋₆ fluoroalkyl), wherein each of the 7-10 membered bicyclic carbocyclyl and 5-10 membered heterocyclyl is optionally further substituted by oxo or oxime, and wherein if the 5-10 membered heterocyclyl contains a suitable ring nitrogen atom, that ring nitrogen may be optionally substituted by C₁₋₃ alkyl.

In some embodiments, a compound is selected from the group consisting of:
6-(3-benzenesulfonamido-2,6-difluorophenyl)-7-fluoro-N-methyl-1H-indazole-3-carboxamide;
6-[2,6-Difluoro-3-[3-(hydroxymethyl)benzenesulfonamido]phenyl]-7-fluoro-N-methyl-1H-indazole-3-carboxamide;
6-[2,6-difluoro-3-[3-fluoro-5-(hydroxymethyl)benzenesulfonamido]phenyl]-7-fluoro-N-methyl-1H-indazole-3-carboxamide;
6-(3-amino-2-fluorophenyl)-7-fluoro-N-methyl-1-[[2-(trimethylsilyl) ethoxy]methyl]indazole-3-carboxamide; N-(6-(3-(5-chloro-2-methoxypyridine-3-sulfonamido)-2,6-difluorophenyl)-7-fluoro-1H-indazol-3-yl) acetamide;
6-[3-[5-chloro-2-(difluoromethoxy)pyridine-3-sulfonamido]-2,6-difluorophenyl]-7-fluoro-N-methyl-1H-indazole-3-carboxamide;
6-[3-[5-Cyano-2-(difluoromethoxy)pyridine-3-sulfonamido]-2,6-difluorophenyl]-7-fluoro-N-methyl-1H-indazole-3-carboxamide;
6-[3-(5-chloro-2-methoxypyridine-3-sulfonamido)-2,6-difluorophenyl]-7-fluoro-N,4-dimethyl-1H-indazole-3-carboxamide;
6-[3-(5-Chloro-2-methoxypyridine-3-sulfonamido)-2,6-difluorophenyl]-7-fluoro-N-(2-methylpropyl)-1H-indazole-3-carboxamide;
6-(3-((5-Chloro-2-methoxypyridine)-3-sulfonamido)-2,6-difluorophenyl)-N-ethyl-7-fluoro-1H-indazole-3-carboxamide;
6-[3-(5-Chloro-2-methoxypyridine-3-sulfonamido)-2,6-difluorophenyl]-7-fluoro-N-(1-methyl-6-oxopiperidin-3-yl)-1H-indazole-3-carboxamide;
N-(1-acetylpyrrolidin-3-yl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-(2-hydroxypropyl)-1H-indazole-3-carboxamide;
6-[3-(5-Chloro-2-methoxypyridine-3-sulfonamido)-2,6-difluorophenyl]-7-fluoro-N-(1-hydroxypropan-2-yl)-1H-indazole-3-carboxamide;
6-[3-(5-Chloro-2-methoxypyridine-3-sulfonamido)-2,6-difluorophenyl]-7-fluoro-N-(2-oxopyrrolidin-3-yl)-1H-indazole-3-carboxamide;
6-[3-(5-Chloro-2-methoxypyridine-3-sulfonamido)-2,6-difluorophenyl]-7-fluoro-N-(oxan-3-yl)-1H-indazole-3-carboxamide;
6-[3-(5-Chloro-2-methoxypyridine-3-sulfonamido)-2,6-difluorophenyl]-7-fluoro-N-(sec-butyl)-1H-indazole-3-carboxamide;
6-[3-(5-Chloro-2-methoxypyridine-3-sulfonamido)-2,6-difluorophenyl]-7-fluoro-N-(pyrrolidin-3-yl)-1H-indazole-3-carboxamide;
6-[3-(5-Chloro-2-methoxypyridine-3-sulfonamido)-2,6-difluorophenyl]-7-fluoro-N-propyl-1H-indazole-3-carboxamide;
6-[3-(5-Chloro-2-methoxypyridine-3-sulfonamido)-2,6-difluorophenyl]-7-fluoro-N-(pentan-2-yl)-1H-indazole-3-carboxamide;
6-[3-(5-chloro-2-methoxypyridine-3-sulfonamido)-2,6-difluorophenyl]-7-fluoro-N-(4-hydroxybutan-2-yl)-1H-indazole-3-carboxamide;
6-[3-(1-Benzofuran-4-sulfonamido)-2,6-difluorophenyl]-7-fluoro-N-methyl-1H-indazole-3-carboxamide;
6-[3-(1H-1,3-Benzodiazole-4-sulfonamido)-2,6-difluorophenyl]-7-fluoro-N-methyl-1H-indazole-3-carboxamide;
6-[2,6-Difluoro-3-(3-methyl-1,3-benzodiazole-4-sulfonamido)phenyl]-7-fluoro-N-methyl-1H-indazole-3-carboxamide;
5-chloro-N-[2,4-difluoro-3-[7-fluoro-3-(1,3-oxazol-2-yl)-1H-indazol-6-yl]phenyl]-2-methoxypyridine-3-sulfonamide;
6-[2,6-difluoro-3-(5-fluoro-2-methylpyridine-3-sulfonamido)phenyl]-7-fluoro-N-methyl-1H-indazole-3-carboxamide;
6-[2,6-Difluoro-3-(5-fluoro-2-methylpyridine-3-sulfonamido)phenyl]-7-fluoro-N-methyl-1H-pyrazolo[4,3-c]pyridine-3-carboxamide;
6-[3-(5-Chloro-2-methoxypyridine-3-sulfonamido)-2,6-difluorophenyl]-7-fluoro-N-methyl-1H-pyrazolo[4,3-c]pyridine-3-carboxamide;
N-[2,4-difluoro-3-[7-fluoro-3-(hydrazinecarbonyl)-1H-indazol-6-yl]phenyl]-1-benzofuran-6-sulfonamide;
6-[2,6-Difluoro-3-(1-methyl-1,3-benzodiazole-4-sulfonamido)phenyl]-7-fluoro-N-methyl-1H-indazole-3-carboxamide;
6-[2,6-Difluoro-3-(5-fluoro-2-methoxypyridine-3-sulfonamido)phenyl]-7-fluoro-N-methyl-1H-pyrazolo[4,3-c]pyridine-3-carboxamide;
6-[3-(5-Cyano-2-methoxypyridine-3-sulfonamido)-2,6-difluorophenyl]-7-fluoro-N-methyl-1H-pyrazolo[4,3-c]pyridine-3-carboxamide;
6-[3-(5-Chloro-2-methylpyridine-3-sulfonamido)-2,6-difluorophenyl]-7-fluoro-N-methyl-1H-pyrazolo[4,3-c]pyridine-3-carboxamide;
5-chloro-N-[2,4-difluoro-3-(7-fluoro-1H-indazol-6-yl)phenyl]-2-methoxypyridine-3-sulfonamide;
6-[3-(cyclopentylmethanesulfonamido)-2,6-difluorophenyl]-7-fluoro-N-methyl-1H-indazole-3-carboxamide;
6-[2,6-difluoro-3-(oxane-4-sulfonamido)phenyl]-7-fluoro-N-methyl-1H-indazole-3-carboxamide;
6-[2,6-Difluoro-3-(6-fluoro-1-hydroxy-2,3-dihydro-1H-indene-4-sulfonamido)phenyl]-7-fluoro-N-methyl-1H-indazole-3-carboxamide;
6-[2,6-difluoro-3-(1-methylpiperidine-3-sulfonamido)phenyl]-7-fluoro-N-methyl-1H-indazole-3-carboxamide;
6-[2,6-difluoro-3-[(3-hydroxycyclopentyl) methanesulfonamido]phenyl]-7-fluoro-N-methyl-1H-indazole-3-carboxamide;
6-[3-(5-Chloro-2-methoxypyridine-3-sulfonamido)-2,6-difluorophenyl]-N-cyclopentyl-7-fluoro-1H-indazole-3-carboxamide;
6-[3-(5-Chloro-2-methoxypyridine-3-sulfonamido)-2,6-difluorophenyl]-7-fluoro-N-(3-hydroxycyclohexyl)-1H-indazole-3-carboxamide;
6-[3-(5-Chloro-2-methoxypyridine-3-sulfonamido)-2,6-difluorophenyl]-N-cyclopropyl-7-fluoro-1H-indazole-3-carboxamide;
6-[3-(5-Chloro-2-ethoxypyridine-3-sulfonamido)-2,6-difluorophenyl]-7-fluoro-N-(morpholin-4-ylmethyl)-1H-indazole-3-carboxamide;
6-[3-(5-chloro-2-methoxypyridine-3-sulfonamido)-2,6-difluorophenyl]-7-fluoro-N-[2-(pyrrolidin-1-yl)ethyl]-1H-indazole-3-carboxamide;
6-[3-(5-Chloro-2-methoxypyridine-3-sulfonamido)-2,6-difluorophenyl]-7-fluoro-N-(2-methanesulfonylethyl)-1H-indazole-3-carboxamide;
6-[3-(5-Chloro-2-methoxypyridine-3-sulfonamido)-2,6-difluorophenyl]-N-(cyclopropylmethyl)-7-fluoro-1H-indazole-3-carboxamide;
6-[3-(5-Chloro-2-methoxypyridine-3-sulfonamido)-2,6-difluorophenyl]-7-fluoro-N-[2-(morpholin-4-yl)ethyl]-1H-indazole-3-carboxamide;
6-[3-(5-Chloro-2-methoxypyridine-3-sulfonamido)-2,6-difluorophenyl]-7-fluoro-N-(2-hydroxycyclopentyl)-1H-indazole-3-carboxamide;
6-[3-(5-Chloro-2-methoxypyridine-3-sulfonamido)-2,6-difluorophenyl]-7-fluoro-N-(2-hydroxycyclohexyl)-1H-indazole-3-carboxamide;
6-[3-(5-Chloro-2-methoxypyridine-3-sulfonamido)-2,6-difluorophenyl]-7-fluoro-N-(2-methylbut-3-yn-2-yl)-1H-indazole-3-carboxamide;
6-[3-(5-Chloro-2-methoxypyridine-3-sulfonamido)-2,6-difluorophenyl]-7-fluoro-N-(2,2,2-trifluoroethyl)-1H-indazole-3-carboxamide;
6-[3-(5-Chloro-2-methoxypyridine-3-sulfonamido)-2,6-difluorophenyl]-N-[cyano (cyclopropyl)methyl]-7-fluoro-1H-indazole-3-carboxamide;
6-[3-(5-Chloro-2-methoxypyridine-3-sulfonamido)-2,6-difluorophenyl]-7-fluoro-N-(prop-2-yn-1-yl)-1H-indazole-3-carboxamide;
6-[3-(5-Chloro-2-methoxypyridine-3-sulfonamido)-2,6-difluorophenyl]-7-fluoro-N-(piperidin-3-ylmethyl)-1H-indazole-3-carboxamide;
N-(2-Aminocyclohexyl)-6-[3-(5-chloro-2-methoxypyridine-3-sulfonamido)-2,6-difluorophenyl]-7-fluoro-1H-indazole-3-carboxamide;
N-(1-Aminopropan-2-yl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-(4-oxocyclohexyl)-1H-indazole-3-carboxamide;
6-[3-(5-Chloro-2-methoxypyridine-3-sulfonamido)-2,6-difluorophenyl]-7-fluoro-N-(3-hydroxycyclopentyl)-1H-indazole-3-carboxamide;
N-(3-Aminocyclohexyl)-6-[3-(5-chloro-2-methoxypyridine-3-sulfonamido)-2,6-difluorophenyl]-7-fluoro-1H-indazole-3-carboxamide;
6-[3-(5-Chloro-2-methoxypyridine-3-sulfonamido)-2,6-difluorophenyl]-7-fluoro-N-[2-(2-oxopyrrolidin-1-yl)ethyl]-1H-indazole-3-carboxamide;
6-[3-(5-Chloro-2-methoxypyridine-3-sulfonamido)-2,6-difluorophenyl]-7-fluoro-N-[(1-methylpyrrolidin-3-yl)methyl]-1H-indazole-3-carboxamide;
6-[3-(1,3-dihydro-2-benzofuran-4-sulfonamido)-2,6-difluorophenyl]-7-fluoro-N-methyl-1H-indazole-3-carboxamide;
6-[3-(2,3-dihydro-1-benzofuran-6-sulfonamido)-2,6-difluorophenyl]-7-fluoro-N-methyl-1H-indazole-3-carboxamide;
6-[3-(2,3-Dihydro-1-benzofuran-6-sulfonamido)-2,6-difluorophenyl]-7-fluoro-N-methyl-1-[[2-(trimethylsilyl) ethoxy]methyl]indazole-3-carboxamide;
6-[3-(6-cyano-1-hydroxy-2,3-dihydro-1H-indene-4-sulfonamido)-2,6-difluorophenyl]-7-fluoro-N-methyl-1H-indazole-3-carboxamide; and
6-[2,6-difluoro-3-(6-fluoro-1-hydroxy-2,3-dihydro-1H-indene-4-sulfonamido)phenyl]-7-fluoro-N-methyl-1H-pyrazolo[4,3-c]pyridine-3-carboxamide,
   or a pharmaceutically acceptable salt, stereoisomer or tautomer thereof.

For example, in some embodiments, the GCN2 inhibitor is selected from the group consisting of the following structures:

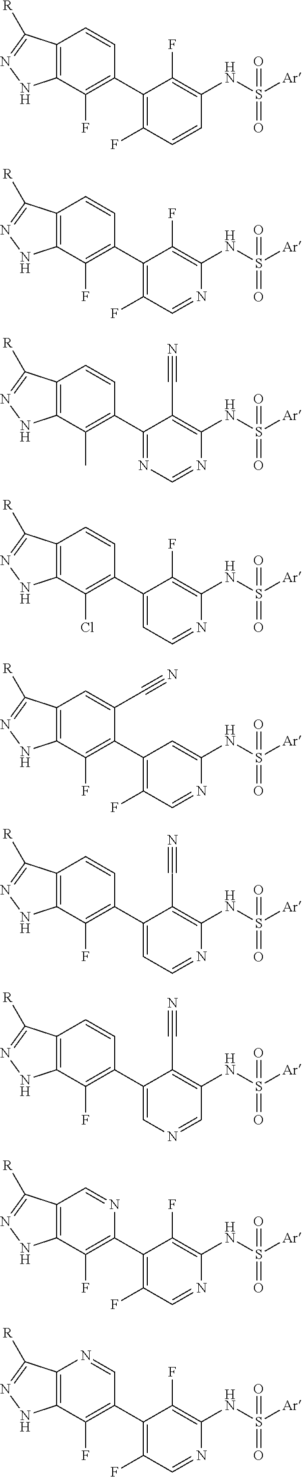

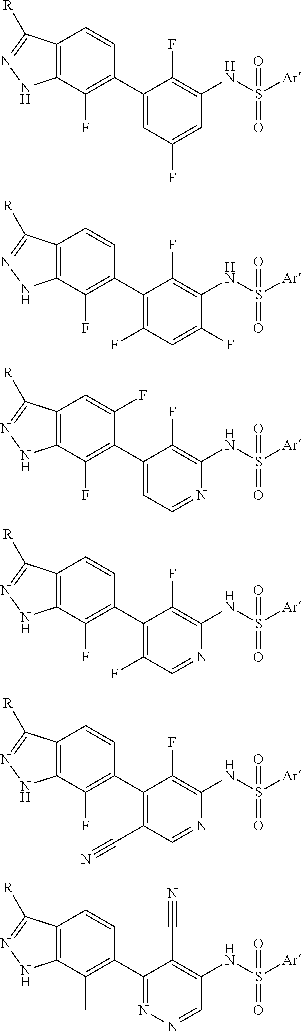

or a pharmaceutically acceptable salt, stereoisomer or tautomer thereof, wherein R and Ar′ are, respectively, A¹ and A³ as defined in formula (V).

In some embodiments, the GCN2 inhibitor is a compound described in U.S. Pat. No. 10,696,651 (WO2018/030466), which is incorporated herein by reference in its entirety. In some embodiments, the GCN2 inhibitor is a compound of Formula (VI):

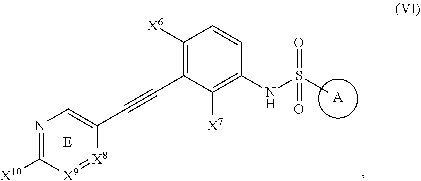

or a pharmaceutically acceptable salt, stereoisomer or tautomer thereof, wherein: ring A is the formula:

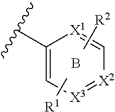

wherein ring B is an optionally further substituted 6-membered aromatic ring;
the combination of X¹, X² and X³ (X¹, X², X³) is (carbon atom, carbon atom, carbon atom) or (carbon atom, carbon atom, nitrogen atom);
R¹ is a (1) a chlorine atom, (2) a bromine atom, (3)methyl, (4)trifluoromethyl, or (5) a hydroxy group substituted by methyl or trifluoromethyl;
R² is (1) a halogen atom, (2) a C₁₋₆ alkyl group optionally substituted by 1 to 3 substituents selected from the group consisting of a halogen atom and a hydroxy group, (3) a C₁₋₆ alkoxy group optionally substituted by 1 to 3 halogen atoms, or the formula:

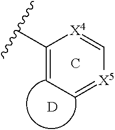

wherein ring C is a 6-membered aromatic ring optionally further substituted by 1 to 3 halogen atoms;
ring D is a 5- to 7-membered non-aromatic heterocycle;
one of X⁴ and X⁵ is a carbon atom, and the other one is a carbon atom or a nitrogen atom;
X⁶ is a hydrogen atom, a fluorine atom, or a chlorine atom;
X⁷ is a fluorine atom or a chlorine atom;
ring E is a nitrogen-containing 6-membered aromatic ring optionally further substituted by 1 to 3 C₁₋₆ alkyl groups;
the combination of X⁸ and X⁹ (X⁸, X⁹) is (carbon atom, nitrogen atom), (nitrogen atom, carbon atom) or (nitrogen atom, nitrogen atom); and
X¹⁰ is an amino group optionally substituted by 1 to 2 substituents selected from the group consisting of (1) a C₁₋₆ alkyl group optionally substituted by 1 to 3 hydroxy groups, (2) a C₃₋₁₀ cycloalkyl group optionally substituted by 1 to 3 hydroxy groups and (3) a 3- to 14-membered non-aromatic heterocyclic group, or
X⁹ and X¹⁰ are bonded to each other to form an unsubstituted 5- to 14-membered aromatic heterocycle.

In some embodiments, the GCN2 inhibitor is

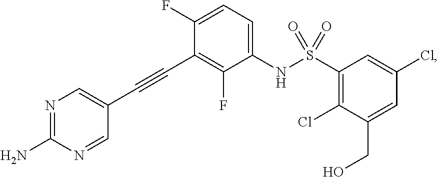 [SMILES: Nc1ncc(C#Cc2c(F)ccc(NS(=O)(=O)c3cc(Cl)cc(CO)c3Cl)c2F)cn1]

or a pharmaceutically acceptable salt, stereoisomer or tautomer thereof.

In some embodiments, the GCN2 inhibitor of formula (VI) is administerend in combination with an amino acid depleting enzymes. In some embodiments, the GCN2 inhibitor of formula (VI) is administered in combination with an Asparaginase.

In some embodiments, the GCN2 inhibitor is a compound described in U.S. Patent Publication No. US20210128563 (WO2017220477), which is incorporated herein by reference in its entirety. In some embodiments, the GCN2 inhibitor is a compound of Formula (VII):

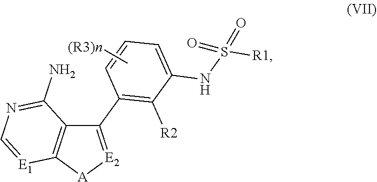

or a pharmaceutically acceptable salt, stereoisomer or tautomer thereof, wherein: n is 0, 1 or 2;
R1 is an optionally substituted group selected from straight or branched (C₁-C₈)alkyl, (C₂-C₈)alkenyl, (C₂-C₈)alkynyl, (C₃-C₈) cycloalkyl, (C₃-C₈) cycloalkenyl, heterocyclyl, aryl and heteroaryl;
R2 and R3 are independently halogen, cyano, OR4 or an optionally substituted group selected from straight or branched (C₁-C₈)alkyl, (C₂-C₈)alkenyl, (C₂-C₈)alkynyl and (C₃-C₈) cycloalkyl, wherein
   R⁴ is an optionally substituted group selected from straight or branched (C₁-C₈)alkyl,
   (C₂-C₈)alkenyl, (C₂-C₈)alkynyl and (C₃-C₈) cycloalkyl;
E1 and E2 are independently CH or N;
A is O, S or NR5, wherein
   R5 is hydrogen or an optionally substituted group selected from straight or branched (C₁-C₈)alkyl, (C₂-C₈)alkenyl, (C₂-C₈)alkynyl, (C₃-C₈) cycloalkyl, (C₃-C₈) cycloalkenyl, heterocyclyl, aryl and heteroaryl.

In some embodiments, the GCN2 inhibitor of formula (VII) is administered in combination with a proteasome inhibitor.

In some embodiments, the GCN2 inhibitor is HCl-1046.

In some embodiments, the GCN2 inhibitor is selected from the group consisting of Compound 1, 2, 3, 4, 5, 6, 7, and 8 from Example A, or a pharmaceutically acceptable salt, stereoisomer, or tautomer thereof.

### V. Kits

Also provided herein are kits comprising a GCN2 inhibitor and instructions for detecting a urea cycle enzyme. In some embodiments, the kit comprises instructions for detecting ASS1. In some embodiments, the kit comprises instructions for detecting the level of ASS1. In some embodiments, the kit comprises instructions for detecting the activity of ASS1. In some embodiments, the kit comprises a nucleic acid probe for ASS1. In some embodiments, the kit comprises primers that hybridize to an ASS1 sequence or an ASS1 regulatory sequence. In some embodiments, the GCN2 inhibitor is any of the molecules described herein.

In some embodiments, the kit comprises an antibody that binds to ASS1. In some embodiments, the antibody is detectably labeled. In some embodiments, the antibody is fluorescently labeled. In some embodiments, the kit comprises a secondary antibody that binds to ASS1.

In some embodiments, the kit further comprises instructions or an agent for detecting a second urea cycle enzyme. In some embodiments, the kit comprises instructions or an agent for detecting three, four, or five enzymes in the urea cycle.

In some embodiments, the kit provides a control sample or control data. In some embodiments, the control sample is non-cancerous cells or tissue. In some embodiments, the kit provides instructions for detecting the level or activity of ASS1 in normal tissue.

In some embodiment, the kit comprises instructions or an agent for detecting an intermediate in the urea cycle. In some embodiments, the kit comprises instructions or an agent for detecting argininosuccinate or citrulline.

In some embodiments, the kit provides instructions for selecting an individual for treatment with a GCN2 inhibitor. In some embodiments, the selection is based upon the level and/or activity) of a urea cycle enzyme (such as ASS1). In some embodiments, the individual is selected for treatment if the cancer has a low level of ASS1 expression or activity. In some embodiments, the instructions provide that the GCN2 inhibitor is administered if the level of the urea cycle enzyme (such as ASS1) is below a certain threshold.

### VI. Formulations

While it is possible for the active compound to be administered alone, it is preferable to present it as a pharmaceutical composition (e.g. formulation).

Accordingly, in some embodiments of the invention, there is provided a pharmaceutical composition comprising at least a compound described herein, for example, a compound of Formula (I) as defined above together with at least one pharmaceutically acceptable excipient.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing the compounds described herein, including compounds of the Formula (I), can be formulated in accordance with known techniques, see for example, Remington's Pharmaceutical Sciences, Mack Publishing Company, Easton, PA, USA. The pharmaceutical compositions can be in any form suitable for oral, parenteral, intravenous, intramuscular, intrathecal, subcutaneous, topical, intranasal, intrabronchial, sublingual, buccal, ophthalmic, otic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

The composition may be a tablet composition or a capsule composition. Tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95%, preferably % (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragées, powders, tablets or capsules.

Tablets and capsules may contain, for example, 0-20% disintegrants, 0-5% lubricants, 0-5% flow aids and/or 0-99% (w/w) fillers/or bulking agents (depending on drug dose). They may also contain 0-10% (w/w)polymer binders, 0-5% (w/w) antioxidants, 0-5% (w/w)pigments. Slow release tablets would in addition typically contain 0-99% (w/w) release-controlling (e.g. delaying)polymers (depending on dose). The film coats of the tablet or capsule typically contain 0-10% (w/w)polymers, 0-3% (w/w)pigments, and/or 0-2% (w/w)plasticizers.

The composition may be a parenteral composition. Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The pharmaceutical formulations may be presented to a patient in “patient packs” containing an entire course of treatment in a single package, usually a blister pack.

The compounds described herein will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### EXAMPLES

### Example A

This example demonstrates that CHOP assay can be used to test the specificity of exemplary Compounds 1-8 towards GCN2 and PERK in cells. The invention is illustrated, but not limited, by reference to the following example compounds shown in Table 1.

| Compound | Structure |
|---|---|
| Compound 1 | 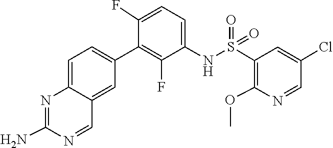 [SMILES: COc1ncc(Cl)cc1S(=O)(=O)Nc1ccc(F)c(-c2ccc3nc(N)ncc3c2)c1F] |
| Compound 2 | 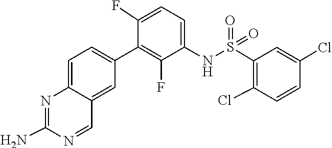 [SMILES: Nc1ncc2cc(-c3c(F)ccc(NS(=O)(=O)c4cc(Cl)ccc4Cl)c3F)ccc2n1] |
| Compound 3 | 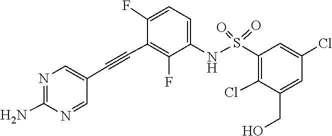 [SMILES: Nc1ncc(C#Cc2c(F)ccc(NS(=O)(=O)c3cc(Cl)cc(CO)c3Cl)c2F)cn1] |
| Compound 4 | 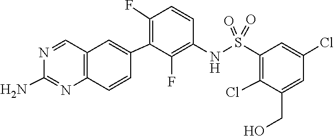 [SMILES: Nc1ncc2cc(-c3c(F)ccc(NS(=O)(=O)c4cc(Cl)cc(CO)c4Cl)c3F)ccc2n1] |
| Compound 5 | 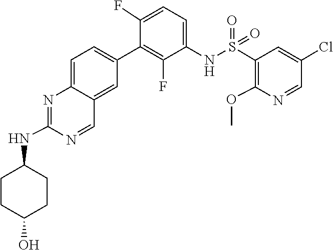 [SMILES: COc1ncc(Cl)cc1S(=O)(=O)Nc1ccc(F)c(-c2ccc3nc(N[C@H]4CC[C@H](O)CC4)ncc3c2)c1F] |
| Compound 6 | 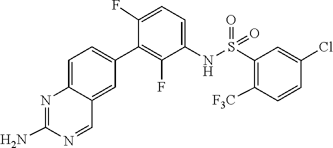 [SMILES: Nc1ncc2cc(-c3c(F)ccc(NS(=O)(=O)c4cc(Cl)ccc4C(F)(F)F)c3F)ccc2n1] |
| Compound 7 | 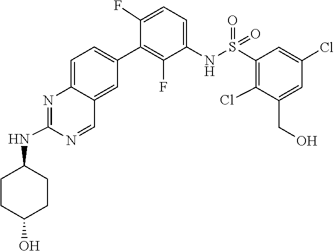 [SMILES: O=S(=O)(Nc1ccc(F)c(-c2ccc3nc(N[C@H]4CC[C@H](O)CC4)ncc3c2)c1F)c1cc(Cl)cc(CO)c1Cl] |
| Compound 8 | 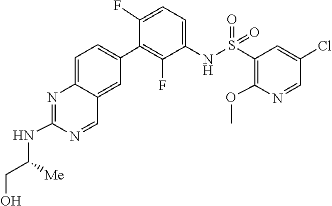 |

Wild type (WT) murine 3T3 fibroblasts stably transduced with a CHOP (DDIT3) reporter construct were evaluated for cell growth (e.g., proliferation). The CHOP construct comprised the 3′ untranslated region (UTR) of mouse DDIT3 mRNA fused to a sequence encoding the fluorescent protein mCherry.

Cells were seeded at 5,000 cells per well in flat-bottom 96-well plates in 100 uL DMEM low glucose media without phenol red and supplemented with 10% FBS and 2 mM glutamine. After 24 hours, the exemplary compounds, and either 200 nM tunicamycin or 100 nM halofuginone, were added together. Cells were placed in live-cell imaging and analysis platform (IncuCyte ZOOM®; Essenbio) set up in a humidified incubator at 37° C. and the plates were scanned every 4 hours for up to 48 hours, at 10× magnification, to determine CHOP induction. Experiments were performed in duplicate with 3 images collected per well and analyzed using live-cell imaging and analysis software (IncuCyte ZOOM® 2018A software; Essenbio).

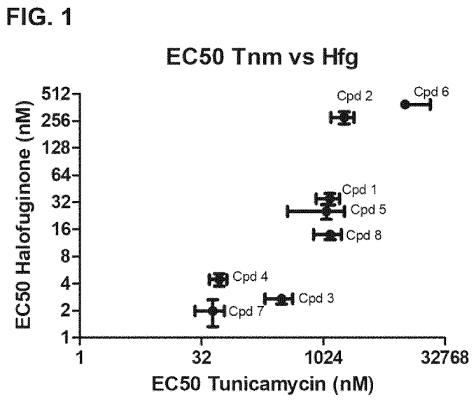

Treatment of the cells with halofuginone induced GCN2 activation, treatment with tunicamycin induced PERK activation. Both lead to an induction of CHOP (mCherry). Thus CHOP can be induced by both GCN2 and PERK (FIG. 1).

Treatment of the cells with increasing concentrations of example compounds reduced GCN2 and/or PERK activation, in a concentration-dependent manner. Shown as the EC50 value of at least 3 independent experiments (FIG. 1). This decrease in overall GCN2/PERK activation by the exemplary compounds resulted in a corresponding decrease of GCN2-induced CHOP activation, in the presence of halofuginone or tunicamycin (FIG. 1).

These results suggest that exemplified compounds target GCN2, with different levels of cross-reactivity on PERK.

### Example B

Validation of the GCN2-mediated mechanism of action with two different cellular reporter systems.

To further demonstrate the action of the exemplary compounds 1-8 the activity of CHOP reporter to ATF4 reporter was compared.

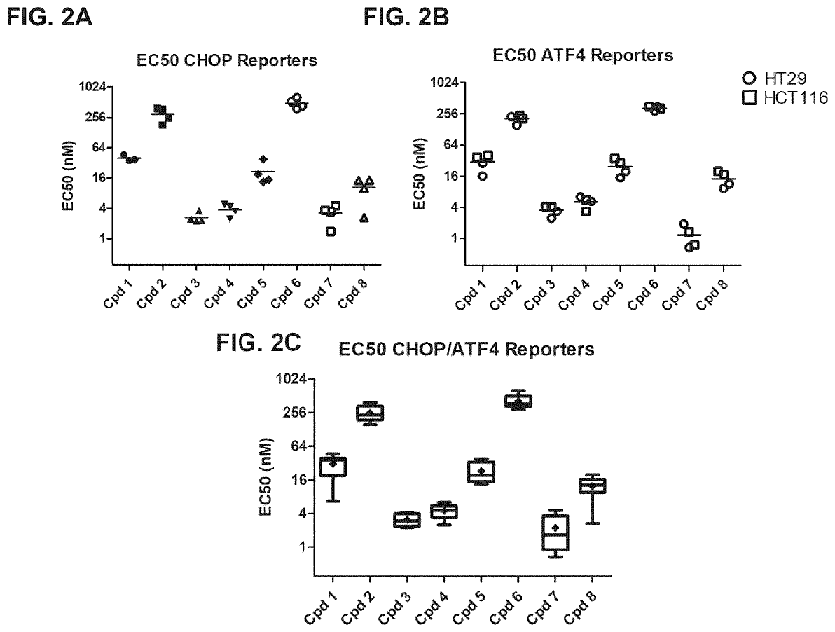

CHOP reporter experiments were performed on halofuginone treated cells as described in the EXAMPLE A. Shown are EC50 values of four independent experiments (FIG. 2A).

For the ATF4 reporter, HT29 and HCT116 colon carcinoma cell lines were stably transduced with an ATF4 reporter construct, comprised of the 5′ UTR of human ATF4 fused to GFP.

Cells were seeded at 5,000 cells per well in flat-bottom 96-well plates in 100 uL DMEM low glucose media without phenol red and supplemented with 10% FBS and 2 mM glutamine. After 24 hours, the exemplary compounds and halofuginone (100 nM), were added together. Cells were placed in live-cell imaging and analysis platform (IncuCyte S3®; Essenbio) set up in a humidified incubator at 37° C. and the plates were scanned every 4 hours for up to 48 hours, at 10× magnification, to determine ATF4 expression. Experiments were performed in triplicate with 4 images collected per well and analyzed using live-cell imaging and analysis software (IncuCyte S3® 2020B software; Essenbio) set up for cell-by-cell analysis.

Treatment of the cells with halofuginone induced GCN2 activation, leading to an induction of the ATF4 signal (GFP). Shown are the EC50 values of exemplary compounds 1-8 (FIG. 2B).

EC50 values from both reporters were combined showing experimental reproducibility (FIG. 2C).

### Example C

This example demonstrates that exemplary compounds 1 and 2 prevent the growth of ASS1-low tumor spheroids.

HT29 and HCT116 cells were maintained at 37° C. and 5% CO₂ in DMEM/F12 medium (Fisher Scientific) supplemented with 10% fetal bovine serum and 1% penicillin-streptomycin 5 (complete medium). 300,000 cells/well were seeded in flat-bottom 6 well plates 48 hours before the experiment.

Each well was washed with sterile 1×PBS and then 100 μL of ice-cold lysis buffer supplemented with phosphatase and protease inhibitors was added. Lysates were transferred to 1.5 mL tubes and sonicated for 3 min, and total protein amounts were quantified using 15 BCA assay (e.g., BCA Protein Assay Kit, Pierce) according to manufacturer instructions. An equal amount of proteins was loaded on SDS-PAGE gels for electrophoresis and then transferred onto 0.2 mm filter pore PVDF membranes.

Membranes were then blocked in blocking buffer containing 3% BSA/0.1% Tween/1× Tris-buffered saline (TBS) for 1 hour and then incubated overnight at 4° C. with agitation with anti-ASS1 primary antibody (Sigma Aldrich), diluted 1:1,000 in blocking buffer. Anti-β actin antibody (Sigma Aldrich) was used as a loading control.

Membranes were then incubated with the corresponding secondary antibody to detect immune-reactive bands using enhanced chemiluminescence (e.g., ECL Western Blotting Substrate Pierce). Images were acquired (e.g., using Chemidoc™ 25 equipment) and quantitated by densitometry using ImageJ software.

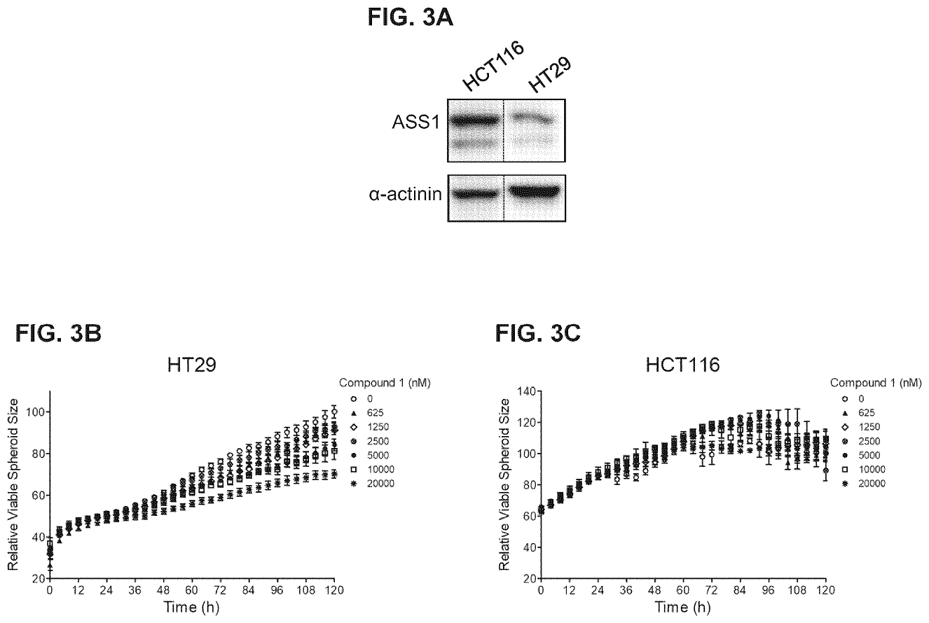

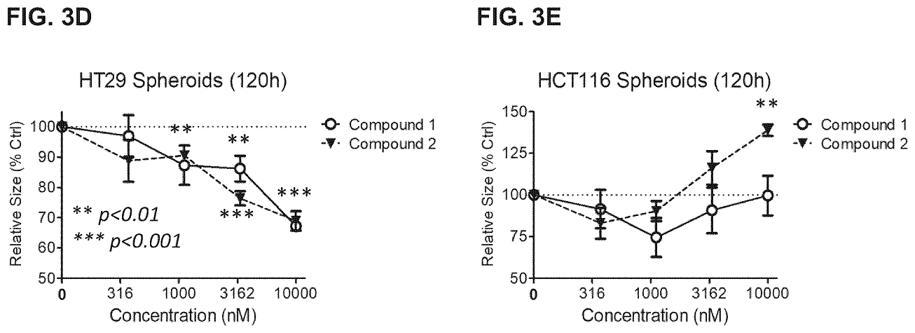

Relative ASS1 expression in ASS1-low HT29 cells and ASS1-high HCT116 cells is shown on FIG. 3A.

The growth of tumor spheroids was evaluated following treatment of cells with Compound 1, Compound 2. Briefly, HT29 or HCT116 cells were seeded at 5,000 cells/well in ultra-low attachment round-bottom 96-well plates, in 100 μL SILAC DMEM Flex media without phenol red, supplemented with 10% fetal bovine serum (FBS), 1 g/L glucose, 2 mM glutamine, 220 μM lysine, and 64 μM arginine. The plates were centrifuged for 10 minutes at 125×g and put in a humidified incubator at 37° C. for 3 days before the onset of the experiment to allow spheroids to form.

Three days after seeding, 0-20 μM Compound 1, 0-20 μM Compound 2, or 10 μM vehicle control (0.1% DMSO), were added together with the Nuclear-ID Red DNA Stain (1:100,000; ENZO Life Sciences) to detect live cells. Plates were placed in live-cell imaging and analysis platform (e.g., IncuCyte ZOOM®; 15 Essenbio) set up in a humidified incubator at 37° C. and scanned every 4 hours for up to 120 hours, at 10× magnification, to determine the relative spheroid size and cell death. Experiments were performed in quadruplicate and analyzed using live-cell imaging and analysis software (e.g., IncuCyte ZOOM® 2018A software; Essenbio).

As demonstrated in FIGS. 3B and 3D, treating the ASS1-low, HT29 spheroids with Compound 1 or 2 significantly reduces spheroid growth in a dose-dependent manner as compared to the DMSO-treated spheroids.

As demonstrated in FIGS. 3C and 3E, treating the ASS1-high HCT116 spheroids with Compound 1 or 2 does not reduce spheroid growth.

### Example D

This example demonstrates EC50 values of selected compounds on the HT29 spheroid growth.

ASS1-low HT29 spheroids were grown and treated with compounds as described in Example D. Briefly, HT29 or HCT116 cells were seeded at 5,000 cells/well in ultra-low attachment round-bottom 96-well plates, in 100 μL SILAC DMEM Flex media without phenol red, supplemented with 10% fetal bovine serum (FBS), 1 g/L glucose, 2 mM glutamine, 220 μM lysine, and 64 μM arginine. The plates were centrifuged for 10 minutes at 125× g and put in a humidified incubator at 37° C. for 3 days before the onset of the experiment to allow spheroids to form.

Three days after seeding, 0-20 μM Compound 1, 0-20 μM Compound 2, or 10 μM vehicle control (0.1% DMSO), were added together with the Nuclear-ID Red DNA Stain (1:100,000; ENZO Life Sciences) to detect live cells. Plates were placed in live-cell imaging and analysis platform (e.g., IncuCyte ZOOM®; 15 Essenbio) set up in a humidified incubator at 37° C. and scanned every 4 hours for up to 120 hours, at 10× magnification, to determine the relative spheroid size and cell death. Experiments were performed in quadruplicate and analyzed using live-cell imaging and analysis software (e.g., IncuCyte ZOOM® 2018A software; Essenbio).

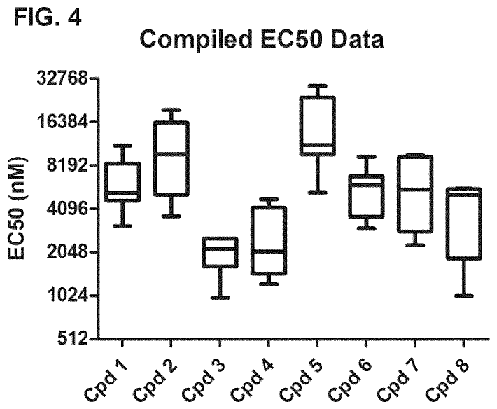

Shown are boxplots of EC50 values from 3 independent experiments calculated using GraphPad Prism (FIG. 4).

### Example E

This example demonstrates the effectiveness of Compounds 1 and 2 in ASS1-low renal cell carcinoma (RCC) tumors.

A498 (ASSIlow) and 769-P (ASSIhigh) cells were seeded at a density of 300,000 cells/well were seeded in in flat-bottom 6 well in RPMI medium with 16 μM arginine, 1 g/L glucose, 2 mM L-glutamine, and 10% FCS and allowed grow for 48 hours before the experiment.

Each well was washed with sterile 1× PBS and then 100 μL of ice-cold lysis buffer supplemented with phosphatase and protease inhibitors was added. Lysates were transferred to 1.5 mL tubes and sonicated for 3 min, and total protein amounts were quantified using 15 BCA assay (e.g., BCA Protein Assay Kit, Pierce) according to manufacturer instructions. An equal amount of proteins was loaded on SDS-PAGE gels for electrophoresis and then transferred onto 0.2 mm filter pore PVDF membranes.

Membranes were then blocked in blocking buffer containing 3% BSA/0.1% Tween/1× Tris-buffered saline (TBS) for 1 hour and then incubated overnight at 4° C. with agitation with anti-ASS1 primary antibody (Sigma Aldrich), diluted 1:1,000 in blocking buffer. Anti-β actin antibody (Sigma Aldrich) was used as a loading control.

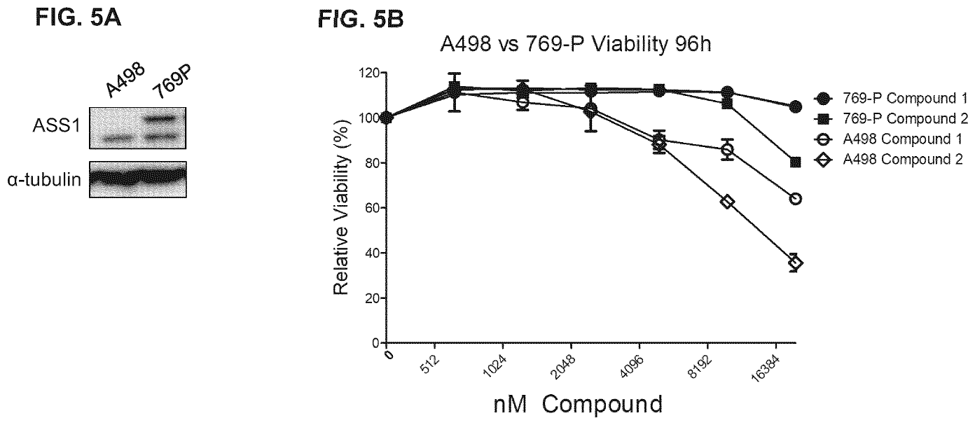

Relative ASS1 expression in ASS1-low A⁴⁹⁸ cells and ASS1-high 769-P cells is shown on FIG. 5A.

To assess the effectiveness of Compound 1 and Compound 2: A⁴⁹⁸ (ASS1low) and 769-P (ASS1high) cells were seeded at a density of 2000 cells/well in 96-well plates in RPMI medium with 16 μM arginine, 1 g/L glucose, 2 mM L-glutamine, and 10% FCS and allowed to adhere overnight.

Cells were then treated with increasing amounts of the indicated compounds for 4 days. Afterward, cell viability was determined by staining the nuclei of the living cells with NUCLEAR-IDR Red DNA stain (Enzo Life Sciences) and the apoptotic/death cells with Annexin V CF488A (Biotium) and scanning the plates with an IncuCyte S3 instrument. Viable cells were classified as Nuclear-ID Red high and Annexin V CF488A low and relative viability was expressed as a percentage of the total number of cells/well.

As demonstrated in FIG. 5B, treating the A498 (ASSIlow) cells with Compound 1 or 2 significantly reduces relative cell viability in a dose-dependent manner. While treating the 769-P (ASS1high) cells with Compound 1 or 2 did not significantly relative cell viability. This demonstrates that cancer cells that express a low level of ASS1 respond significantly better to GCN2 inhibitors than those that express a high level of ASS1.

### Example F

Further experiments are performed on isogenic cancer cells that differ only with respect to ASS1 level (i.e. the same cell line with expressing a high level of ASS1 and expressing a low level of ASS1). The purpose of these experiments is to test whether a low level of a urea cycle enzyme (such as ASS1) level by itself is responsible for tumor sensitivity to GCN2.

Cells are be grown as monolayers or in a 3D culture using physiological medium conditions. These cells are then be treated with increasing concentrations of test compounds. Cell proliferation and/or survival are be monitored using optical or biochemical methods (e.g.: live-cell imaging and analysis platform (IncuCyte ZOOM®; Essenbio). Based on the in vitro results—in vivo experiments are be performed. Tumors are grown in the appropriate mouse strain. Then the tumors are treated with the test compound. Tumor growth and biomarkers including, but not limited to urea cycle status are assessed.

### Example G

The growth of tumor spheroids was evaluated following treatment with Compound 2. Briefly, HT29 cells were seeded at 2,000 cells/well and HCT116 cells were seeded at 1,000 cells/well in ultra-low attachment round bottom 96-well plates in soft agar with 100 μl SILAC DMEM flex media without phenol red, supplemented with 10% Dialyzed fetal bovine serum (dFBS), 1 g/l glucose, 2 mM glutamine, 220 μM Lysine, and 64 μM arginine. Various concentrations of compound 2 (1.37 nM, 4.11 nM, 12.3 nM, 37 nM, 111 nM, 333 nM, and 1000 nM) were added. Plates were placed in a humidified incubator at 37° C. and incubated for 5 days, allowing colonies to form. At endpoint, images were taken by light microscopy (3 images per well) and analyzed using ImageJ software.

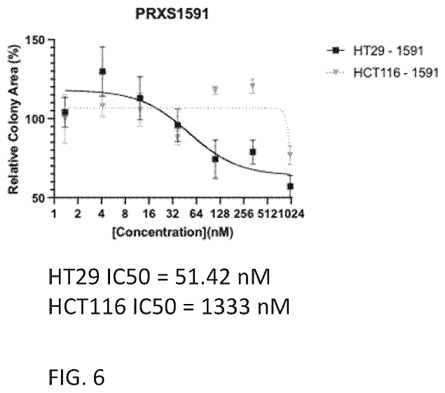

FIG. 3A shows that HCT116 cells have a higher level of ASS1 expression than HT29 cells. As shown in FIG. 6, strong growth inhibition by compound 2 was observed in HT29 cells (IC50 52.42 nM). In contrast, growth inhibition of HCT116 cells was only observed at high levels of compound 2 (IC50 1333 nM). Accordingly, this data show that compounds of the present invention can inhibit growth of tumor cells that express high levels of ASS1.

### Example H

The viability of cancer cell lines was evaluated following treatment with compound 2. Briefly, 769P and A498 cells were seeded at 100-6,400 cells/well in 96 well plates in 100 μl SILAC DMEM flex media without phenol red, supplemented with 10% dialyzed fetal bovine serum (dFBS), 1 g/l glucose, 2 mM glutamine, 220 μM Lysine, and 64 μM arginine. The cells were put in a humidified incubator at 37° C. for 24 hours. The next day compounds were added at various concentrations. At the end of the experiment, ATPlite 1 Step™ (Perkin Elmer) solution was added to each well and shaken for 2 minutes. After incubation of 10 min in the dark, luminescence was recorded on a Envision Multimode Reader to determine cell viability.

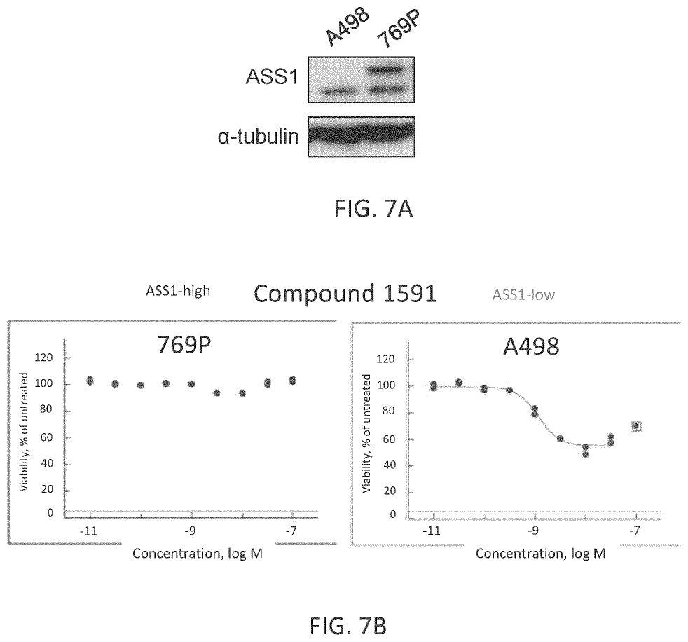

FIG. 7A is a western blot showing expression of ASS1 in A498 and 769P cells with tubulin control. As show in FIG. 7A, A498 cells express a low level of ASS1 in comparison to 769P cells. Incubation of A498 cells with compound 2 resulted in a significant level of cell death, as shown in FIG. 7B.

### Example I

EdU incorporation levels of cancer cell lines was evaluated following treatment with compound 1, compound 2, compound 3, or vehicle control (0.1% DMSO). Briefly, HT29 and HCT116 cells were seeded at 2,500 cells/well in 96 well plates in 100 μl SILAC DMEM flex media without phenol red, supplemented with 10% dialyzed fetal bovine serum (dFBS), 1 g/l glucose, 2 mM glutamine, 220 μM Lysine, and various concentrations of arginine (6.25 μM, 12.5 μM, 25 μM, and 100 μM). The cells were put in a humidified incubator at 37° C. for 24 hours. The next day compounds were added to the cells at a concentration of 1 M. At day 3, cells were incubated with 10 μM EdU for 24 hours. The next day EdU positive cells were labeled using the Click-it reaction cocktail per manufacturing protocol using Amplex UltraRed Dye. Fluorescence was measured using a microplate reader using filter sets appropriate for Amplex™ UntraRed reagent (ex 568 nm and em 585 nm).

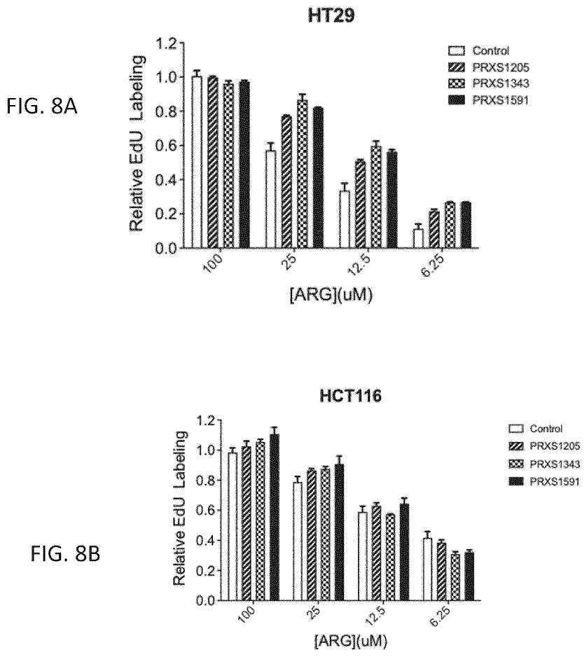

FIG. 8A shows that in HT29 cells that express a low level of ASS1, Compounds 1, 2 and 3 cause significantly higher levels of EdU labeling. In contrast, as shown in FIG. 8B, in HCT116 cells that express a high level of ASS1, Compounds 1, 2 and 3, have a smaller effect on EdU labeling. This suggests that the GCN2 inhibitors, Compounds 1, 2, and 3, modulate the cell cycle. Without wishing to be bound by theory, Compounds 1, 2, and 3 may cause premature release into S phase resulting in tumor cell death.

### Example J

Effect of GCN2 inhibition on in vivo xenograft tumor models of various ASS1 low cancer models was evaluated following treatment with compound 1, compound 2, or vehicle control. Briefly, 1,000,000 tumor cells with a total volume of 100 μl were injected into the rear flank region of 6-8 week old BALB/c or C₅₇BL/6 female mice. The following tumor cell lines were tested: renica (renal adenocarcinoma), CT26 (colon cancer), MC 38 (colon adenocarcinoma), B16F10 (melanoma) When tumors reached a size of approximately 100 mm2, mice were treated orally (PBS, 10 μl/g, i.p., twice weekly (BIW); Compound 1: 30 mg/kg, 10 μl/g, p.o., twice daily (BID); or Compound 2: 20 mg/kg, 10 μl/g, p.o., BID). Tumor volumes were measured every 3-4 days. Endpoint measurements as presented were measured at day 20 (Renca), day 14 (CT26), day 17 (MC38), and day 11 (B16F10).

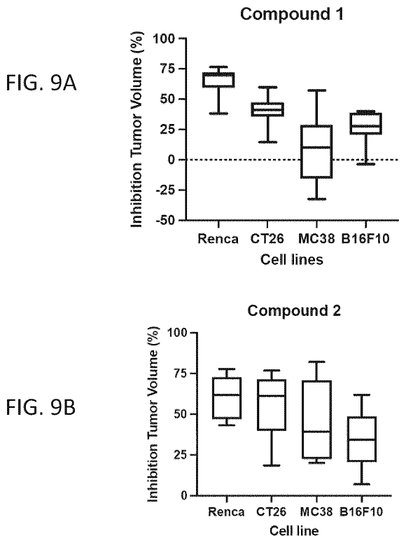

As shown in FIGS. 9A and 9B Compound 1 and Compound 2 caused a significant inhibition in tumor volume compared to control across a variety of tumor cells. For example, as shown in FIG. 9A, compound 1 caused a 60-70% reduction in tumor volume compared to control.

### Example K

ASS1 RNA levels were compared from primary solid tumor samples with normal tissue (liver) from the TCGA public database.

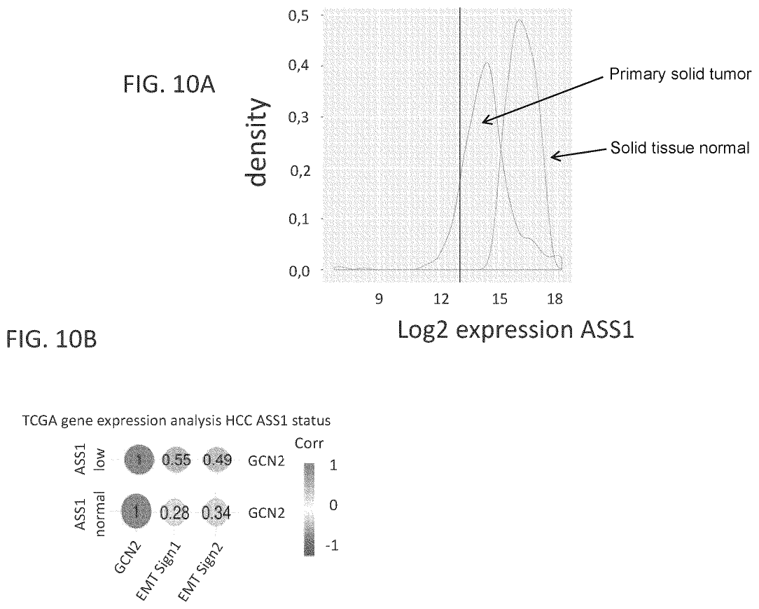

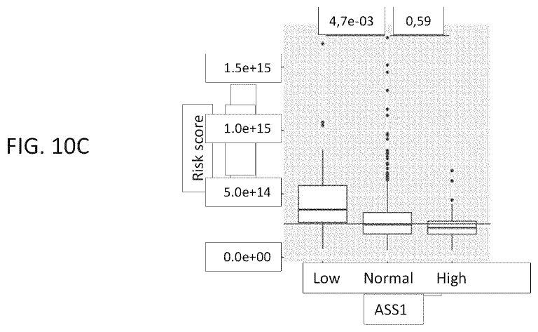

As shown in FIG. 10A, ASS1 mRNA level is significantly lower in solid tumors compared to normal tissues, suggesting that ASS1 is silenced in tumor cells.

Expression data from the Cancer Genome Atlas (TCGA) was used to determine whether ASS1 status correlates to aggressiveness of hepatocellular carcinoma (HCC). For this analysis ASS1 low cells comprise the lowest quartile of cells expressing ASS1. As shown in FIG. 10B, ASS1 low status correlates with two markers of epithelial to mesenchymal transition (EMT) shown as EMTSign1 and EMTSign2.

Survival analysis reveals that comparing the 25% lowest ASS1 expressing tumors have a significant poorer survival compared to the 25% highest ASS1 expressing tumors. See FIG. 1I of Missiaen et al., Cell Metabolist 34:1-17 (Aug. 2, 2022). Using TCGA data, the autophagy risk of ASS1 low, ASS1 normal, or ASS1 high cancers was analyzed. Similar to the analysis above, ASS1 low cancers are the lowest quartile of ASS1 expression, ASS1 high cancers are the highest quartile of ASS1 expression. ASS1-low expressing cancers have a significant increase in the autophagy risk score, indicating that they are actively undergoing autophagy, as shown in FIG. 10C.

Taken together these data support that low ASS1 cancers tend to be more aggressive resulting in decreased survival rates of individuals with low ASS1 cancers.

### SUMMARY OF THE INVENTION

The present invention describes the use of GCN2 inhibitors for the treatment of cancers with low levels of expression or activity of urea cycle enzyme(s). Related methods of detection and diagnosis are also provided.

1. A method of treating a cancer in an individual comprising administering a GCN2 inhibitor to the individual, wherein the cancer has a low level of expression or activity of a urea cycle enzyme.

2. A method of treating cancer in an individual comprising
a. detecting the level of expression or activity of a urea cycle enzyme, and
b. administering a GCN2 inhibitor to the individual if the cancer expresses a low level of expression or activity of a urea cycle enzyme.

3. A method of predicting responsiveness to a GCN2 inhibitor in an individual having cancer comprising detecting the level of expression or activity of a urea cycle enzyme in the cancer, wherein if the cancer has a low level of expression or activity of a urea cycle enzyme, the cancer is responsive to the GCN2 inhibitor.

4. A method of inhibiting tumor growth in an individual comprising administering a GCN2 inhibitor to the individual, wherein the tumor has a low level of expression or activity of a urea cycle enzyme.

5. A method of inhibiting cell proliferation in an individual comprising administering a GCN2 inhibitor to the individual, wherein the cell has a low level of expression or activity of a urea cycle enzyme.

6. The method of claim 1, wherein the urea cycle enzyme is ASS1.

7. The method of claim 6, wherein the cancer, tumor, or cell expresses a low level of ASS1 protein or mRNA.

8. The method of claim 1, further comprising comparing the level of expression or activity of ASS1 to a control.

9. The method of claim 8, wherein the cancer, tumor, or cell has at least 1.5 fold lower expression of ASS1 than the control.

10. The method of claim 8, wherein the control is a sample is obtained from non-cancerous tissue of the same origin as the cancer, tumor, or cell.

11. The method of claim 8, wherein the control is the average expression level of ASS1 level derived from a population of subjects.

12. The method of claim 1, wherein the GCN2 inhibitor is selected from the group consisting of Compounds 1-8.

13. The method of claim 1, wherein the GCN2 inhibitor decreases the activity of PERK.

14. The method of claim 13, wherein the activity of PERK is decreased at least 1.5 fold.

15. The method of claim 1, wherein the GCN2 inhibitor decreases the level of CHOP.

16. The method of claim 15, wherein the level of CHOP is decreased at least 1.5 fold.

17. The method of claim 1, wherein the GCN2 inhibitor does not decrease the activity of PERK.

18. The method of claim 1, wherein the GCN2 inhibitor does not decrease the level of CHOP.

19. The method of claim 1, wherein the cancer is a solid or hematological tumor.

20. The method of claim 1, wherein the cancer is selected from the group consisting of breast cancer, colorectal cancer, ovarian cancer, prostate cancer, pancreatic cancer, kidney cancer, lung cancer, melanoma, fibrosarcoma, bone sarcoma, connective tissue sarcoma, renal cell carcinoma, giant cell carcinoma, squamous cell carcinoma, leukemia, skin cancer, soft tissue cancer, liver cancer, gastrointestinal carcinoma, adenocarcinoma, hepatocellular carcinoma, thyroid cancer, multiple myeloma, cancer of secretory cells, myelodysplastic syndrome, myeloproliferative neoplasm, malignant glioma, non-Hodgkin's lymphoma, Hodgkin's lymphoma, Burkitt's lymphoma, chronic lymphocytic leukemia, chronic myeloid leukemia, hairy cell leukemia, monoclonal gammopathy of undetermined significance (MGUS), plasmacytoma, lymphoplasmacytic lymphoma, acute lymphoblastic leukemia, acute myeloid leukemia, chronic myelomonocytic leukemia, juvenile myelomonocytic leukemia, large granular lymphocytic leukemia, B-cell prolymphocytic leukemia, T-cell prolymphocytic leukemia, small cell lung cancer, malignant pleural mesothelioma, Head and neck squamous cell carcinoma, glioblastoma multiforme, sarcoma, or pediatric neuroblastoma.

21. The method of claim 1, wherein the cancer is selected from hepatocellular carcinoma (HCC), renal cell carcinoma (RCC), and colorectal cancer (CRC).

22. The method of claim 1, wherein the level of expression of the urea cycle enzyme is determined by measuring mRNA level or protein level.

23. The method of claim 22, wherein the level of ASS1 protein or mRNA is measured by western blot or immunohistochemistry, qPCR, FISH, nanostring, or RNAseq.

24. The method of claim 1, wherein the level of ASS1 protein or mRNA expressed by the cancer is lower than the level of ASS1 protein expressed in non-cancer cells or less responsive cancers.

25. The method of claim 1, wherein the tumor environment comprises a low level of arginine.

26. The method of claim 1, wherein the individual is a human.

27. The method of claim 1, wherein the GCN2 inhibitor is a compound of formula (I), (I-1), (I-2), (II), (IIa), (III) or (IV), a pharmaceutically acceptable salt, stereoisomer or tautomer thereof.

28. The method of claim 1, wherein the method comprises administering to the individual an effective amount of the GCN2 inhibitor, or a pharmaceutically acceptable salt, stereoisomer or tautomer thereof.

29. A kit comprising a GCN2 inhibitor and instructions for detecting the level of a urea cycle enzyme.

30-35. (canceled)
